(19) United States
(12) Patent Application Publication
(10) Pub. No.: US 2012/0288515 A1
(43) Pub. Date: Nov. 15, 2012

(54) SYNTHETIC LONG PEPTIDE (SLP)-BASED VACCINES

(75) Inventors: Scott H. Robbins, Lake Forest Park, WA (US); Thomas W. Dubensky, JR., Seattle, WA (US)

(73) Assignee: IMMUNE DESIGN CORP., Seattle, WA (US)

(21) Appl. No.: 13/458,098

(22) Filed: Apr. 27, 2012

Related U.S. Application Data
(60) Provisional application No. 61/479,611, filed on Apr. 27, 2011.

(51) Int. Cl. A61K 39/00; A61P 35/00; A61P 31/04; A61K 39/245; A61P 31/12

(52) U.S. Cl. 424/186.1; 424/185.1

(57) ABSTRACT

Synthetic long peptides and an adjuvant are formulated together and administered to a subject in order to raise an immune response. In certain embodiments, the adjuvant is GLA.

This invention was made with U.S. government support under grant numbers 5-R43-AI087444-02 awarded by the National Institutes of Health and National Institute of Allergy and Infectious Disease. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 46442A_SeqListing.txt. The text file is 24,576 bytes, was created on Apr. 26, 2012, and is being submitted electronically via EFS-Web.

### TECHNICAL FIELD

The present invention relates generally to vaccine compositions and immunotherapy for human and veterinary use. In particular, the present invention relates to compositions comprising synthetic long peptides and an adjuvant for use as a therapeutic or prophylactic vaccine and methods of making and using the compositions.

### BACKGROUND

Many diseases have been essentially eradicated or are well-controlled though vaccination of the world's population. Children in the United States are vaccinated against more than a dozen diseases such as measles, polio and tetanus. Most of these vaccines comprise heat-killed or disabled organisms as the immunizing agent. A few vaccines contain just the proteins from the disease-causing organism. In such cases, the proteins are either purified from the organisms or are made by recombinant DNA methods. For examples, tetanus vaccine contains heat-inactivated toxin from Clostridium and hepatitis B vaccine contains one of the viral envelope proteins, hepatitis B surface antigen (HBsAg), which is produced by yeast cells transformed with the gene encoding HBsAg.

All of these vaccine types have disadvantages that limit their usefulness. Organisms may be grown in cultures that contain allergens, which can cause anaphylactic shock in susceptible individuals (e.g., influenza virus grown in chicken eggs); heat-inactivation of an organism or protein may be incomplete or sufficiently destroy the immunogen so that it doesn't raise a vigorous immune response; it may be difficult to scale production to meet world-wide demand.

A totally synthetic vaccine would have many advantages. In terms of inspiring public confidence, contaminants would be virtually eliminated as would allergic reactions. Production could be more easily scaled up, and wouldn't require biologically secure facilities, thus opening up vaccine production to more countries.

Synthetic vaccines based on synthetic long peptides (SLPs) have been used in the development of vaccines to prevent gynecological cancers. Two clinical trials involving SLPs representing human papillomavirus (HPV)-16 E6 and E7 oncoproteins in one case, and the p53 protein in the other, were performed in vulver intraepithelial neoplasia and ovarian cancer patients, respectively (Kenter, et al., N. Engl. J. Med. 361:1838-1847, 2009; Leffers, et al., Int. J. Cancer 125:2104-2113, 2009) A major disadvantage to these SLP vaccines is that they must be delivered in extremely high quantities in their current formulations with the adjuvant Montanide, which makes the cost-of-goods a very prohibitive factor.

Described herein are SLP vaccine compositions in improved formulations that overcome the disadvantages of prior vaccines formulations and provide additional other advantages.

### SUMMARY

In one aspect, the invention provides an immunogenic composition comprising one or more SLPs (synthetic long peptides) and an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons. In various embodiments, where any two or more embodiments may be combined, the invention further provides: wherein the one or more SLPs are present at a concentration wherein an increase in the concentration results in a decrease in the immunogenicity of the composition; the composition comprises from about 0.1 μg to about 15 μg of each SLP; the one or more SLPs are derived from an HSV-2 protein; SLPs are derived from UL19; one or more SLPs are derived from a cancer antigen, e.g., the cancer antigen is NY-ESO-1, TRP2, or CAIX; the adjuvant is GLA; the immunogenic composition is aqueous and oil-free or comprises less than about 1% v/v oil; the immunogenic composition is prepared from an adjuvant which was formulated as a stable oil-in-water emulsion.

In another aspect, the invention provide a method of immunizing a subject to provide a therapeutic or prophylactic effect, against cancer or infectious agents such as HSV-2, comprising administering a immunogenic composition comprising one or more SLPs derived from an HSV-2 protein and an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons. In various embodiments, where any two or more embodiments may be combined, the invention further provides: a method wherein the adjuvant is GLA and the HSV-2 protein is UL19; a method wherein a second (boosting) composition comprising an immunogen is administered following (priming) administration of the immunogenic composition and wherein the immunogen is recombinantly produced.

These and other aspects will become evident upon reference to the following detailed description and attached drawings.

### BRIEF DESCRIPTION OF THE DRAWINGS

FIG. 1 presents graphs showing the percentage of CD4+ and CD8+ T-cells generated in response to immunization with UL19 SLPs in varying amounts.

FIG. 2 shows CD8/CD4 T-cell responses directed against SIV-Gag can be observed when SIV-Gag is delivered at varying doses of SLP.

FIG. 3 shows CD8/CD4 T-cell responses directed against SIV-Gag can be observed when SIV-Gag is delivered at varying doses of SLP and that an adjuvant is necessary to observe these responses.

FIG. 4 shows adjuvant is required during the prime and the boost of an immunization regimen to generate antigen specific CD4 T-cell responses.

### DETAILED DESCRIPTION

The present disclosure provides compositions for use as vaccines and methods of immunizing subjects with the vaccines for therapeutic or prophylactic treatment, in which the vaccines comprise a synthetic long peptide and an adjuvant. The synthetic long peptides comprise at least one of a CD4 epitope or a CD8 epitope or at least one each of a CD4 and CD8 epitope.

### 1. Synthetic Long Peptides

“Synthetic long peptide” (SLP) refers to a protein sequence manufactured ex vivo and having a length as short as about 25 amino acids and as long as about 100 amino acids. An SLP should be long enough to be taken up and processed by dendritic cells for presentation on their cell surface with MHC class I or class II molecules. It is understood that SLPs may also be recombinantly produced. For example, it is contemplated that peptides comprising at least one CD4 epitope or at least one CD8 epitope or at least one CD4 and at least one CD8 epitope, and at least 25 amino acids and as long as 100 amino acids in length, as described below for SLPs, can be produced recombinantly (i.e., “RLP”) and purified using known techniques. Thus, the description herein of SLPs also applies to RLPs.

An SLP comprises at least one CD4 epitope or at least one CD8 epitope or at least one CD4 and at least one CD8 epitope. A CD4 epitope refers to an amino acid sequence that binds to class II MHC and a CD8 epitope refers to an amino acid sequence that binds to class I MHC. Epitope sequences are derived from the amino acid sequence of an immunogen; in vivo, briefly, the immunogen is taken up or synthesized by antigen-processing cells (e.g., dendritic cells) and degraded into peptides, which associate with MHC molecules and are presented on the cell surface as an MHC-peptide complex. Peptides complexed with MHC class I molecules interact with the T-cell antigen receptor and CD8 on CD8+ T-cells, these peptides are called CD8 epitopes; peptides complexed with MHC class II molecules interact with T-cell antigen receptor and CD4 on CD4+ T-cells, these peptides are called CD4 epitopes. Activated CD8+ T-cells become cytotoxic T-cells, which recognize and kill targeT-cells displaying the MHC class I-CD8 epitopes. Often, targeT-cells are infected or tumor cells. Activated CD4+ T-cells become helper T-cells, and depending on their subtype, help B cells to produce antibody or activate natural killer cells, phagocytes and CD8+ T-cells. Activation of both CD4+ T-cells and CD8+ T-cells contribute to a comprehensive cellular immune response.

As disclosed above, an SLP should be long enough to be taken up and processed by dendritic cells and presented on their cell surface with MHC molecules. Peptides complexed with MHC class I molecules are generally 8-11 amino acids in length, and peptides complexed with MHC class II molecules are generally 13-17 amino acids in length, although longer or shorter lengths are not uncommon. In various embodiments, an SLP will be between 40-50, 35-55, 30-60, 25-65 or 20-70 amino acids in length. An SLP will typically be at least 25 amino acids long and as long as 100 amino acids long (e.g., at least 30 aa, at least 35 aa, at least 40 aa, at least 45 aa, at least 50 aa, at least 55 aa, at least 60 aa, at least 65 aa, at least 70 aa, at least 75 aa, at least 80 aa, at least 85 aa, at least 90 aa, at least 95 aa). It is understood that the SLP may be also up to 30 aa, up to 35 aa, up to 40 aa, up to 45 aa, up to 50 aa, up to 55 aa, up to 60 aa, up to 65 aa, up to 70 aa, up to 75 aa, up to 80 aa, up to 85 aa, up to 90 aa, or up to 95 aa in length. Any of these lengths may be combined with any other lengths to form all possible ranges without having to set forth each combination herein. The length of an SLP will generally be about 45 aa or about 50 aa in length.

Epitopes may have known sequence or unknown sequence. A plethora of proteins have been mapped for CD4 and CD8 epitopes. For SLPs comprising one or more of these epitopes, the length will typically be about 45 aa. Moreover, the epitope may be flanked by about 15 aa at the N-terminal and at the C-terminal sides. The flanking sequences are typically the sequences that flank the epitope sequence in the native protein. As discussed above, an SLP may comprise more than one epitope, the multiple epitopes may be all CD4 or CD8 epitopes or a mixture of CD4 and CD8 epitopes. Furthermore, the epitopes may overlap in sequence (see Example 1 for some exemplary SLPs that comprise overlapping epitopes). The total number of SLPs used may be such that all known CD4 and CD8 epitopes are represented.

When a protein has not been mapped for CD4 epitopes or CD8 epitopes or both, a set of SLPs that comprise the entire protein sequence may be synthesized. Each SLP will typically be about 50 aa, and consecutive SLPs may overlap in sequence by about 25 aa. Alternatively, or in addition, algorithms and computer programs can be used to predict sequences that will bind to MHC class I and class II molecules. Such programs are readily available, e.g., RANKPEP (Reche et al., Human Immunol 63: 701, 2002), Epipredict (Jung et al., Biologicals 29: 179, 2001) and MHCPred (Guan et al. Nucl Acids Res 31: 3621, 2003 and Guan et al., Appl Bioinformatics 5: 55, 2006), EpiMatrix (EpiVax, Inc.).

The sequence of an SLP may be adjusted as necessary for optimum production. For example, one or more amino acids at the ends of a peptide derived from a native sequence may be omitted in order to improve solubility or stability, or to increase or decrease the overall charge. As a specific example, a peptide sequence with a high content of hydrophobic amino acids may be difficult to solubilize. As a guide, hydrophobic content is ideally less than 50%. Peptides containing cysteine, methionine, or tryptophan residues, especially multiple Cys, Met, or Trp residues, may be difficult to synthesize. Substitution of another amino acid, either a standard or a non standard amino acid, such as hydroxyproline, gamma-aminobutyric acid, norleucine, may improve synthesis efficiency or purity. Other considerations in designing an SLP include the extent of β-sheet formation, N-terminal amino acid (e.g., an N-terminal Gln can cyclize), minimizing adjacent Ser and Pro residues.

SLPs may be synthesized by any of a variety of methods (see Corradin et al., Sci Translational Med 2:1, 2010 for a general discussion of synthesis methods). Automated peptide synthesizers are commercially available, and many companies provide synthesis services (e.g., Abbiotec, American Peptide Company, AnaSpec, Bachem, Covance Research Products, Invitrogen). Following synthesis, peptides are purified, typically by HPLC, although alternative purification methods such as ion exchange chromatography and gel filtration chromatography may be used. Acceptable purity is at least 90% or at least 95% or at least 98% as assessed by analytical HPLC.

SLPs may be stored in dry form (e.g., lyophilized) or as an aqueous solution. Lyophilized peptides are stored preferably at temperatures of −20° C. or lower. Peptides in solution are stored in sterile, purified water, which may also contain salts, buffers, preservatives or other additives that assist in facilitating dissolution of the peptide. Lyophilized peptides are generally dissolved first in sterile distilled or deionized water. To increase the rate of dissolution, sonication may be performed. Peptide dissolution may be improved by lowering or raising the pH of the solution, depending on whether the peptide is basic or acidic. If peptides are frozen for storage, it is generally advisable to aliquot the peptides in one-use amounts to avoid freeze-thaw cycles.

### 2. Target Antigens

As discussed herein, SLPs are peptides derived from proteins against which an immune response is desired. In one embodiment, the immune response is a T-cell response. The proteins may be known antigens or, in the case of some proteins, they may be candidate antigens. The proteins may be found in a variety of sources of diseases, including infectious agents, such as bacteria, fungi, and viruses, and cancer cells. Exemplary proteins that may serve as a basis for SLP synthesis include those discussed below.

Illustrative pathogenic organisms whose proteins are contemplated for SLPs for the vaccines of the present disclosure include human immunodeficiency virus (HIV), herpes simplex virus (HSV), Varicella Zoster virus (VZV), cytomegalovirus (CMV), Epstein-Barr virus (EBV), respiratory syncytial virus (RSV), vesicular stomatitis virus (VSV), hepatitis B virus (HBV), hepatitis c virus (HCV), Mycobacterium species included Mycobacterium tuberculosis, Staphylococcus species including Methicillin-resistant Staphylococcus aureus (MRSA), Streptococcus species including Streptococcus pneumonia, and Plasmodium species including Plasmodium falciparum. As would be understood by the skilled person, proteins derived from these and other pathogenic organisms for use in the vaccines described herein are known in the art and may be identified in public databases such as GENBANK, Swiss-Prot and TrEMBL.

SLPs may be derived from human immunodeficiency virus (HIV) proteins for certain embodiments of the present vaccines; the proteins include any of the HIV virion structural proteins gp120, gp41, p17, p24, protease, reverse transcriptase, or HIV proteins encoded by tat, rev, nef, vif, vpr and vpu. HIV proteins are well known to the skilled person and may be found in any of a number of public databases (see e.g., Vider-Shalit T, et al., AIDS. 2009 Jul. 17; 23(11):1311-8; Watkins D I Mem Inst Oswaldo Cruz. 2008 March; 103(2):119-29; F. Gao et al., Expert Rev Vaccines. 2004 August; 3(4 Suppl):S161-8). HIV-Gag is a common target for HIV vaccine candidates. SIV-Gag, although from a simian virus, is used commonly as a model antigen for immunology and vaccines as its function in SIV is homologous to the function of HIV-Gag for HIV.

Proteins derived from herpes simplex virus, especially HSV 1 and 2, that are contemplated include, but are not limited to, proteins expressed from HSV late genes. The late group of genes predominantly encode proteins that form the virion particle. Such proteins include the five proteins from (UL) which form the viral capsid; UL6, UL18, UL35, UL38 and the major capsid protein UL19, all of which may be used in the vaccines of the present disclosure (see e.g., McGeoch D J, et al., (2006) Virus Res. 117 (1): 90-104; Mettenleiter T C, et al., (2006) Curr. Opin. Microbiol. 9 (4): 423-9). All of the references referred to in this section are incorporated herein by reference, in their entirety). Other illustrative HSV proteins contemplated for use herein include the ICP27 (H1, H2), glycoprotein B (gB) and glycoprotein D (gD), UL25, UL46, UL49, ICPO, UL39, and UL29 proteins. There are at least 74 genes in the HSV genome, each encoding a protein that could potentially be a vaccine target.

Antigens derived from Varicella zoster virus (VZV) that are contemplated for use in certain embodiments include the viral glycoproteins: gB, gC, gE, gH, gI, gK, gL, gM and gN, and viral tegument proteins. There are at least 70 genes in the VZV genome, each encoding a protein that could potentially be a vaccine target.

Antigens derived from cytomegalovirus (CMV) that are contemplated for use in certain embodiments include CMV viral structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (B J Rykman, et al., J Virol. 2006 January; 80(2):710-22.), envelope glycoprotein B (gB), gH, gN, and pp150. As would be understood by the skilled person, CMV proteins for use in the vaccines described herein may be identified in public databases such as Swiss-Prot and TrEMBL. (see e.g., Bennekov T, et al., Mt. Sinai J. Med. 71 (2): 86-93 (2004); Loewendorf A and Benedict C A. J Intern Med.; May; 267(5):483-501 (2010); Marschall M, Stamminger T. Future Microbiol. August; 4:731-42, (2009)).

Antigens derived from Epstein-Barr virus (EBV) that are contemplated for use in certain embodiments include EBV lytic proteins gp350 and gp110, EBV proteins produced during latent cycle infection including Epstein-Barr nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see e.g., Lockey T D, et al., Front. Biosci. 13: 5916-27 (2008)).

Antigens derived from respiratory syncytial virus (RSV) that are contemplated for use in certain embodiments include any of the 11 proteins encoded by the RSV genome, or immunogenic fragments thereof: NS1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use in certain embodiments include the five major proteins encoded by its genome, and immunogenic fragments thereof: large protein (L), glycoprotein (G), nucleoprotein (N), phosphoprotein (P), and matrix protein (M) (see e.g., Rieder M, Conzelmann K K. J Interferon Cytokine Res. September; 29(9):499-509 (2009); Roberts A, et al. Adv Virus Res. 53:301-19 (1999)).

Antigens derived from Hepatitis B virus (HBV) that are contemplated for use in certain embodiments include the four major proteins encoded by its genome and immunogenic fragments thereof: HBcAg, HBeAg, DNA polymerase, and HBsAg (see e.g., Chisari, F. V. et al. Annu Rev Immunol. 13:29-60. (1995)).

Antigens derived from Hepatitis C virus (HCV) that are contemplated for use in certain embodiments include the structural protein, core, the envelop proteins, E1 and E2, and the p7 protein, and the nonstructural proteins: NS2, NS3, NS4a, NS4b, NSSa, and NSSb (see, e.g., Abida S. et al., Virol J. 8:55-67 (2011)).

Antigens derived from Staphylococcus species including Methicillin-resistant Staphylococcus aureus (MRSA) that are contemplated for use in certain embodiments of the present vaccines include virulence regulators and immunogenic fragments thereof, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins or immunogenic fragments thereof that may be included in the presently disclosed vaccines include Clp proteins, HtrA, MsrR, aconitase, CcpA, SvrA, Msa, CfvA and CfvB (Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder E E, et al. Nucleic Acids Res. 35 (Database issue): 401-6 (2007). PMID: 17142235). As would be understood by the skilled person, Staphylococcus proteins may also be identified in other public databases such as Swiss-Prot and TrEMBL.

Antigens derived from Streptococcus pneumoniae that are contemplated for use in certain embodiments include pneumolysin, PspA, choline-binding protein A (CbpA), NanA, NanB, SpnHL, PavA, LytA, and pilin proteins (RrgA; RrgB; RrgC) and immunogenic fragments of any of these antigens. Immunogenic proteins of Streptococcus pneumoniae are also known in the art and are contemplated for use in the vaccines of the present invention (see e.g., G. Zysk et al. Infect Immun. June; 68(6):3740-3 (2000)). The complete genome sequence of a virulent strain of Streptococcus pneumoniae has been sequenced (see e.g., Tettelin H, et al., Science. 2001 Jul. 20; 293(5529):498-506) and, as would be understood by the skilled person, Streptococcus proteins for use in the vaccines described herein may also be identified in other public databases such as Swiss-Prot and TrEMBL. Proteins of particular interest for vaccines of the present disclosure include virulence factors and proteins predicted to be exposed at the surface of the bacteria (see e.g., Tettelin H., et al. Supra; C. Frolet et al., BMC Microbiol. July 12; 10:190 2010); D J Rigden, et al. Crit Rev Biochem Mol Biol.;38(2):143-68 (2003); Jedrzejas M J. Microbiol Mol Biol Rev. June; 65(2):187-207 (2001)).

Antigens derived from Plasmodium falciparum that are contemplated for use in certain embodiments include CS, TRAP, MSP1, AMA1, MSP3, EBA, GLURP, RAP1, RAP2, Sequestrin, PfEMP1, Pf332, LSA1, LSA3, STARP, PfEXP1, Pfs25, Pfs28, PFS27125, Pfs16, Pfs48/45, Pfs230.

Antigens derived from Mycobacterium tuberculosis that are contemplated for use in certain embodiments include Th Ra12, Tb H9, Tb Ra35, Tb38-1, Erd 14, DPV, MTI, MSL, mTTC2, hTCC1 (see e.g., WO99/51748, the entire contents of which is herein incorporated by reference)

According to certain embodiments, the vaccine compositions, and related formulations and methods of use, may include SLPs that are derived from cancer cell proteins. The SLP vaccines may be useful for the immunotherapeutic treatment or prevention of cancers. For example, the vaccines herein may find utility when formulated to include SLPs from one or more tumor antigens such as those for prostate, breast, colorectal, lung, pancreatic, renal or melanoma cancers. Exemplary cancer or cancer cell-derived antigens include melanoma associated antigens NY-ESO-1 and TRP-2 and renal cell associated antigen carbonic anhydrase IX (CAIX). NY-ESO-1 (GenBank Accession No: CAA05908, 7 Oct. 2008) is also called LAGE-1 (Lethe et al., Int. J. Cancer 76: 903, 1998). It is an 180 aa protein. TRP-2 (tyrosinase-related protein 2) is expressed in melanocytic cells (Bouchard et al., Eur J. Biochem. 219: 127, 1994). CAIX (GenBank Accession No: Q16790.2) is a 459 amino acid protein that is induced by hypoxia and expressed in renal cell tumors (Klatte et al., Clin. Canc. Res, 13:7388-7393, 2007).

Other cancer antigens include those believed to have high potential of efficacy in cancer therapy (Cheever et al., Hum Cancer Biol 15:5323, 2009; incorporated in its entirety). Based on a number of criteria and characteristics of an ideal cancer antigen, a panel of 75 antigens were deemed as high priority candidates. These include WT1, MUC1, LMP2, HPV E6 and E7, EGFRvIII, HER-2/neu, MAGE A3, wt p53, PSMA, GD2, CEA, MelanA/MART1, Ras mutant, NY-ESO-1, gp100, p53 mutant, Proteinase3, bbcr-ab1, tyrosinase, surviving, PSA, hTERT, Sarcoma translocation breakpoints, EphA2, PAP, ML-IAP, AFP, EPCAM, EFG (TMPRSS2 ETS fusion gene), NA17, PAX3, ALK, androgen receptor, cyclin B1, polysialic acid, MYCN, RhoC, TRP-2, GD3, fucosyl GM1, mesothelin, PSCA, MAGE A1, sLe, CYP1B1, PLAC1, GM3, BORIS, Tn, GloboH, ETV6-AML, NY-BR-1, RGS5, SART3, Stn, PAX5, OY-TES1, sperm protein 17, LCK, HMV/MAA, AKAP-4, SSX2, XAGE 1, B7H3, legumain, Tie2, Page4, VEGF$2, MAD-CT-1, FAP, PDGFR-beta, MAD-CT-2, fos-related antigen 1. The list of 75 antigens is illustrative and is not exhaustive for cancer antigens that may serve as the basis for SLPs. These non-limiting examples of cancer antigens are expressed in a wide range of tumor types such as melanoma, lung carcinoma, sarcoma and bladder carcinoma.

T-cell epitopes in any of the exemplary SLP antigens herein set forth may be identified by any one or combination of methodologies known in the art. By way of example, T-cell epitopes of a designated antigen may be identified using a peptide motif searching program based on algorithms developed by Rammensee, et al. (Immunogenetics 50: 213-219 (1999)); by Parker, et al. (supra), or by using methods such as those described by Doytchinova and Flower in Immunol. Cell Biol. 80(3):270-9 (2002); Blythe et al., Bioinformatics 18:434-439 (2002); Guan et al., Applied Bioinformatics 2:63-66 (2003); Flower et al., Applied Bioinformatics 1:167-176 (2002); Mallios, Bioinformatics 17:942-48 (2001); Schirle et al., J. Immunol. Meth. 257:1-16 (2001).

Epitopic regions of designated microbial antigens or designated tumor antigens are also described in the art. See by way of example, Lamb et al., Rev. Infect. Dis. March-April: Suppl 2:s443-447 (1989); Lamb et al., EMBO J. 6:1245-49 (1987); Lamb et al., Lepr. Rev. Suppl 2:131-37 (1986); Mehra et al., Proc. Natl. Acad. Sci. USA 83:7013-27 (1986); Horsfall et al., Immunol. Today 12:211-13 (1991); Rothbard et al., Curr. Top. Microbiol. Immunol. 155:143-52 (1990); Singh et al., Bioinformatics 17:1236-37 (2001); DeGroot et al., Vaccine 19:4385-95 (2001); DeLalla et al., J. Immunol. 163:1725-29 (1999); Cochlovius et al., J. Immunol. 165:4731-41 (2000); Consogno et al., Blood 101:1039-44 (2003); Roberts et al., AIDS Res. Hum. Retrovir. 12:593-610 (1996); Kwok et al., Trends Immunol. 22:583-88 (2001); Novak et al., J. Immunol. 166:6665-70 (2001).

Additional methods for identifying epitopic regions include methods described in Hoffmeister et al., Methods 29:270-281 (2003); Maecker et al., J. Immunol. Methods 255:27-40 (2001). Assays for identifying epitopes are described herein and known to the skilled artisan and include, for example, those described in Current Protocols in Immunology, Coligan et al. (Eds), John Wiley & Sons, New York, N.Y. (1991).

Identifying an immunogenic region and/or epitope of a designated antigen of interest can also be readily determined empirically by a person skilled in the art and/or by computer analysis and computer modeling, using methods and techniques that are routinely practiced by persons skilled in the art. Empirical methods include, by way of example, synthesizing polypeptide fragments comprising a particular length of contiguous amino acids of a protein, or generating fragments by use of one or more proteases and then determining the immunogenicity of the fragments using any one of numerous binding assays or immunoassay methods routinely practiced in the art. Exemplary methods for determining the capability of an antibody (polyclonal, monoclonal, or antigen-binding fragment thereof) to specifically bind to a fragment include, but are not limited to, ELISA, radioimmunoassay, immunoblot, competitive binding assays, fluorescence activated cell sorter analysis (FACS), and surface plasmon resonance.

Sequences of T-cell epitopes can be obtained from publically available databases. For example, a peptide database that includes T-cell defined tumor antigens can be found on the Internet in a peptide database sponsored by Cancer Immunity (see cancerimmunity(dot)org/peptidedatabase/Tcellepitopes.htm), which is updated periodically. Another available database supported by the National Institute of Allergy and Infectious Diseases, which provides tools for searching for B cell and T-cell epitopes and provides epitope analysis tools (see Immune Epitope Database and Analysis Resource at immunoepitope(dot)org).

In certain instances when antigen-specific T-cell lines or clones are available, for example tumor-infiltrating lymphocytes (TIL), virus-specific or bacteria-specific cytotoxic T lymphocytes (CTL), these cells may be used to screen for the presence of relevant epitopes using targeT-cells prepared with specific antigens. Such targets can be prepared using random, or selected, synthetic peptide libraries, which would be used to sensitize the targeT-cells for lysis by the CTL. Another approach to identify a relevant epitope when T-cell lines or clones are available is to use recombinant DNA methodologies. Gene or cDNA libraries from CTL-susceptible targets are first prepared and transfected into non-susceptible targeT-cells. This allows the identification and cloning of the gene encoding the protein precursor of the peptide containing the CTL epitope. The second step in this process is to prepare truncated genes from the relevant cloned gene, in order to narrow down the region that encodes for the at least one CTL epitope. This step is optional if the gene is not too large. The third step is to prepare synthetic peptides of, for example, approximately 10-20 amino acids in length, overlapping by 5-10 residues, which are used to sensitize targets for the CTL. When a peptide, or peptides, is shown to contain the relevant epitope, and if desired, smaller peptides can be prepared to establish the peptide of minimal size that contains the epitope. These epitopes are typically, but not necessarily, contained within 9-10 residues for CTL epitopes and up to 20 or 30 residues for helper T lymphocyte (HTL) epitopes.

Alternatively, epitopes may be defined by direct elution of peptides that are non-covalently bound by particular major histocompatibility complex (MHC) molecules followed by amino acid sequencing of the eluted peptides (see, for example, Engelhard et al., Cancer J. 2000 May; 6 Suppl 3:S272-80). Briefly, the eluted peptides are separated using a purification method such as HPLC, and individual fractions are tested for their capacity to sensitize targets for CTL lysis or to induce proliferation of cytokine secretion in HTL. When a fraction has been identified as containing the peptide, it is further purified and submitted to sequence analysis. The peptide sequence can also be determined using tandem mass spectrometry. A synthetic peptide is then prepared and tested with the CTL or HTL to corroborate that the correct sequence and peptide have been identified.

Epitopes may also be identified using computer analysis, such as the Tsites program (see, e.g., Rothbard and Taylor, EMBO J. 7:93-100, 1988; Deavin et al., Mol. Immunol. 33:145-155, 1996), which searches for peptide motifs that have the potential to elicit Th responses. CTL peptides with motifs appropriate for binding to murine and human class I or class II MHC may be identified according to BIMAS (Parker et al., J. Immunol. 152:163, 1994) and other HLA peptide binding prediction analyses. Briefly, the protein sequences, for example from microbial components or antigens, or tumor cell components or tumor antigens, are examined for the presence of MHC-binding motifs. These binding motifs, which exist for each MHC allele, are conserved amino acid residues, usually at positions 2 (or 3) and 9 (or 10) for MHC class I binding peptides that are typically 9-10 residues long. Synthetic peptides are then prepared that comprise those sequences bearing the MHC binding motifs, and subsequently such peptides are tested for their ability to bind to MHC molecules. The MHC binding assay can be carried out either using cells which express high numbers of empty (unoccupied) MHC molecules (cellular binding assay), or using purified MHC molecules. Lastly, the MHC binding peptides are then tested for their capacity to induce a CTL response in naive individuals, either in vitro using human lymphocytes, or in vivo using HLA-transgenic animals. These CTL are tested using peptide-sensitized target cells, and targets that naturally process the antigen, such as viral infected cells or tumor cells. To further confirm immunogenicity, a peptide may be tested using an HLA A2 transgenic mouse model and/or any of a variety of in vitro stimulation assays.

### 3. Adjuvant

The present invention provides compositions, kits, methods, etc. which include and/or utilize an adjuvant intended to enhance (or improve, augment) the immune response to a target antigen (e.g., SLP) (i.e., increase the level of the specific immune response to the target antigen in a statistically, biologically, or clinically significant manner compared with the level of the specific immune response in the absence of administering the adjuvant). In other embodiments, instead of combining an adjuvant with the immunogenic composition comprising the target antigen or administering the adjuvant concurrently with this immunogenic composition, the adjuvant is administered at a later time and may be administered by a different route and/or a different site than the immunogenic composition comprising the target antigen. When the adjuvant is administered after administration of the immunogenic composition comprising the target antigen, the adjuvant is administered at 18 hours, 24 hours, 36 hours, 72 hours or 1 day, 2 days, 3 days, 4, days, 5 days, 6 days, or seven days (1 week) after administration of the immunogenic composition. Methods and techniques for determining the level of an immune response are discussed in greater detail herein and are routinely practiced in the art.

Exemplary adjuvants that may be included in the immunogenic compositions and used in the methods described herein include, but are not necessarily limited to, the following. Adjuvants that may be used in these methods include adjuvants useful for enhancing the humoral response, the cellular response, or both the humoral and cellular responses specific for the immunogen(s) and respective designated antigen(s). The cellular immune response comprises a CD4 T-cell response (which may include a memory CD4 T-cell response) and a CD8 T-cell response specific for the immunogen and its respective designated antigen. The cellular response may also include a cytotoxic T-cell response (CTL response) to the target antigen (or to a cell or particle bearing or expressing the target antigen(s)). Desired adjuvants augment the response to the target antigen without causing conformational changes in the target antigen that might adversely affect the qualitative form of the response. Suitable adjuvants include aluminum salts, such as alum (potassium aluminum sulfate), or other aluminum containing adjuvants; nontoxic lipid A-related adjuvants such as, by way of non-limiting example, nontoxic monophosphoryl lipid A (see, e.g., Tomai et al., J. Biol. Response Mod. 6:99-107 (1987); Persing et al., Trends Microbiol. 10:s32-s37 (2002)); GLA described herein; 3 De-O-acylated monophosphoryl lipid A (MPL) (see, e.g., United Kingdom Patent Application No. GB 2220211); adjuvants such as QS21 and QuilA that comprise a triterpene glycoside or saponin isolated from the bark of the Quillaja saponaria Molina tree found in South America (see, e.g., Kensil et al., in Vaccine Design: The Subunit and Adjuvant Approach (eds. Powell and Newman, Plenum Press, NY, 1995); U.S. Pat. No. 5,057,540). Other suitable adjuvants include oil in water emulsions (such as squalene or peanut oil), optionally in combination with immune stimulants, such as monophosphoryl lipid A (see, e.g., Stoute et al., N. Engl. J. Med. 336, 86-91 (1997)). Another suitable adjuvant is CpG (see, e.g., Klinman, Int. Rev. Immunol. 25(3-4):135-54 (2006); U.S. Pat. No. 7,402,572; European Patent No. 772 619).

As described herein, a suitable adjuvant is an aluminum salt, such as aluminum hydroxide, aluminum phosphate, or aluminum sulfate. Such adjuvants can be used with or without other specific immunostimulating agents such as MPL or 3-DMP, QS21, polymeric or monomeric amino acids such as polyglutamic acid or polylysine. Another class of suitable adjuvants is oil-in-water emulsion formulations (also called herein stable oil in water emulsions). Such adjuvants can be optionally used with other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-A1-D-isoglu-L-Ala-dipalmitoxy propylamide (DTP-DPP) theramide™), or other bacterial cell wall components. Oil-in-water emulsions include (1) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.); (2) SAF, containing 10% Squalane, 0.4% Tween 80, 5% pluronic-blocked polymer L121, and thr-MDP, either microfluidized into a submicron emulsion or vortexed to generate a larger particle size emulsion, and (3) Ribi adjuvant system (RAS), (Ribi Immunochem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™). Also as described above, suitable adjuvants include saponin adjuvants, such as Stimulon™(QS21, Aquila, Worcester, Mass.) or particles generated therefrom such as ISCOMs (immunostimulating complexes) and ISCOMATRIX. Other adjuvants include Complete Freund's Adjuvant (CFA) (which is suitable for non-human use but is unsuitable for human use) and Incomplete Freund's Adjuvant (IFA). Other adjuvants include cytokines, such as interleukins (IL-1, IL-2, and IL-12), macrophage colony stimulating factor (M-CSF), and tumor necrosis factor (TNF).

As described herein, an adjuvant may be a non-toxic lipid A-related (or lipid A derivative) adjuvant. In a particular embodiment, an adjuvant is selected on the basis of its capability to act as a Toll-like receptor (TLR) agonist. By way of example, a non-toxic lipid A-related adjuvant that acts as a TLR4 agonist and that may be used in the compositions described herein is identified as DSLP. DSLP compounds share the features that they contain a disaccharide (DS) group formed by the joining together of two monosaccharide groups selected from glucose and amino substituted glucose, where the disaccharide is chemically bound to both a phosphate (P) group and to a plurality of lipid (L) groups. More specifically, the disaccharide may be visualized as being formed from two monosaccharide units, each having six carbons. In the disaccharide, one of the monosaccharides will form a reducing end, and the other monosaccharide will form a non-reducing end. For convenience, the carbons of the monosaccharide forming the reducing terminus will be denoted as located at positions 1, 2, 3, 4, 5 and 6, while the corresponding carbons of the monosaccharide forming the non-reducing terminus will be denoted as being located at positions 1′, 2′, 3′, 4′, 5′ and 6′, following conventional carbohydrate numbering nomenclature. In the DSLP, the carbon at the 1 position of the non-reducing terminus is linked, through either an ether (—O—) or amino (—NH—) group, to the carbon at the 6′ position of the reducing terminus. The phosphate group will be linked to the disaccharide, preferably through the 4′ carbon of the non-reducing terminus. Each of the lipid groups will be joined, through either amide (—NH—C(O)—) or ester (—O—C(O)—) linkages to the disaccharide, where the carbonyl group joins to the lipid group. The disaccharide has 7 positions which may be linked to an amide or ester group, namely, positions 2′, 3′, and 6′ of the non-reducing terminus, and positions 1, 2, 3 and 4 of the reducing terminus.

A lipid group has at least six carbons, preferably at least 8 carbons, and more preferably at least 10 carbons, where in each case the lipid group preferably has no more than 24 carbons, preferably no more than 22 carbons, and more preferably no more than 20 carbons. In one aspect the lipid groups taken together provide 60-100 carbons, preferably 70 to 90 carbons. A lipid group may consist solely of carbon and hydrogen atoms, i.e., it may be a hydrocarbyl lipid group, or it may contain one hydroxyl group, i.e., it may be a hydroxyl-substituted lipid group, or it may contain an ester group which is, in turn, joined to a hydrocarbyl lipid or a hydroxyl-substituted lipid group through the carbonyl (—C(O)—) of the ester group, i.e., a ester substituted lipid. A hydrocarbyl lipid group may be saturated or unsaturated, where an unsaturated hydrocarbyl lipid group will have one double bond between adjacent carbon atoms.

The DSLP comprises 3, or 4, or 5, or 6 or 7 lipids. In one aspect, the DSLP comprises 3 to 7 lipids, while in another aspect the DSLP comprises 4-6 lipids. In one aspect, the lipid is independently selected from hydrocarbyl lipid, hydroxyl-substituted lipid, and ester substituted lipid. In one aspect, the 1, 4′ and 6′ positions are substituted with hydroxyl. In one aspect, the monosaccharide units are each glucosamine. The DSLP may be in the free acid form, or in the salt form, e.g., an ammonium salt.

In one aspect, the lipid on the DSLP is described by: the 3′ position is substituted with —O—(CO)—CH2-CH(Ra)(—O—C(O)—Rb); the 2′ position is substituted with —NH—(CO)—CH2-CH(Ra)(—O—C(O)—Rb); the 3 position is substituted with —O—(CO)—CH2-CH(OH)(Ra); the 2 position is substituted with —NH—(CO)—CH2-CH(OH)(Ra); where each of Ra and Rb is selected from decyl, undecyl, dodecyl, tridecyl, tetradecyl, where each of these terms refer to saturated hydrocarbyl groups. In one embodiment, Ra is undecyl and Rb is tridecyl, where this adjuvant is described in, e.g., U.S. patent application publication 2008/0131466 as “GLA”. The compound wherein Ra is undecyl and Rb is tridecyl may be used in a stereochemically defined form, as available from, e.g., Avanti Polar Lipid as PHAD™ adjuvant.

In another aspect, the DSLP is a mixture of naturally-derived compounds known as 3D-MPL. 3D-MPL adjuvant is produced commercially in a pharmaceutical grade form by GlaxoSmithKline Company as their MPL™ adjuvant. 3D-MPL has been extensively described in the scientific and patent literature, see, e.g., Vaccine Design: the subunit and adjuvant approach, Powell M. F. and Newman, M. J. eds., Chapter 21 Monophosphoryl Lipid A as an adjuvant: past experiences and new directions by Ulrich, J. T. and Myers, K. R., Plenum Press, New York (1995) and U.S. Pat. No. 4,912,094.

In another aspect, the DSLP adjuvant may be described as comprising (i) a diglucosamine backbone having a reducing terminus glucosamine linked to a non-reducing terminus glucosamine through an ether linkage between hexosamine position 1 of the non-reducing terminus glucosamine and hexosamine position 6 of the reducing terminus glucosamine; (ii) an O-phosphoryl group attached to hexosamine position 4 of the non-reducing terminus glucosamine; and (iii) up to six fatty acyl chains; wherein one of the fatty acyl chains is attached to 3-hydroxy of the reducing terminus glucosamine through an ester linkage, wherein one of the fatty acyl chains is attached to a 2-amino of the non-reducing terminus glucosamine through an amide linkage and comprises a tetradecanoyl chain linked to an alkanoyl chain of greater than 12 carbon atoms through an ester linkage, and wherein one of the fatty acyl chains is attached to 3-hydroxy of the non-reducing terminus glucosamine through an ester linkage and comprises a tetradecanoyl chain linked to an alkanoyl chain of greater than 12 carbon atoms through an ester linkage. See, e.g., U.S. patent application publication 2008/0131466.

In another aspect, the adjuvant may be a synthetic disaccharide having six lipid groups as described in U.S. patent application publication 2010/0310602.

In another aspect, the adjuvant used in the present invention may be identified by chemical formula (1):

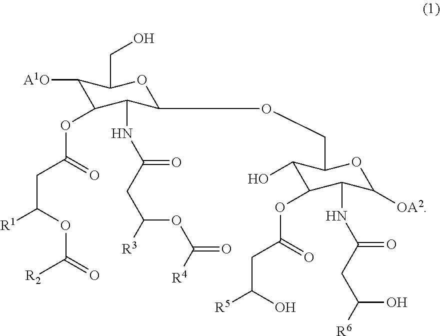

In chemical formula (1), the moieties A¹ and A² are independently selected from the group of hydrogen, phosphate, and phosphate salts. Sodium and potassium are exemplary counterions for the phosphate salts. The A¹O— group, which is preferably a phosphate group, is bonded to the disaccharide at the 4′ position of the non-reducing terminus. The non-reducing terminus is bonded through its 1 position to an ether group, which in turn is bonded to the 6′ position of the reducing terminus. The compounds of chemical formula (1) have six lipid groups that each incorporate one of the moieties R¹, R², R³, R⁴, R⁵, and R⁶, where these R groups are independently selected from the group of hydrocarbyl having 3 to 23 carbons, represented by C₃-C₂₃. For added clarity it will be explained that when a moiety is “independently selected from” a specified group having multiple members, it should be understood that the member chosen for the first moiety does not in any way impact or limit the choice of the member selected for the second moiety. The carbon atoms to which R¹, R³, R⁵ and R⁶ are joined are asymmetric, and thus may exist in either the R or S stereochemistry. In one embodiment all of those carbon atoms are in the R stereochemistry, while in another embodiment all of those carbon atoms are in the S stereochemistry.

“Hydrocarbyl” refers to a chemical moiety formed entirely from hydrogen and carbon, where the arrangement of the carbon atoms may be straight chain or branched, noncyclic or cyclic, and the bonding between adjacent carbon atoms maybe entirely single bonds, i.e., to provide a saturated hydrocarbyl, or there may be double or triple bonds present between any two adjacent carbon atoms, i.e., to provide an unsaturated hydrocarbyl, and the number of carbon atoms in the hydrocarbyl group is between 3 and 24 carbon atoms. The hydrocarbyl may be an alkyl, where representative straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while branched alkyls include isopropyl, sec-butyl, isobutyl, tert-butyl, isopentyl, and the like. Representative saturated cyclic hydrocarbyls include cyclopropyl, cyclobutyl, cyclopentyl, cyclohexyl, and the like; while unsaturated cyclic hydrocarbyls include cyclopentenyl and cyclohexenyl, and the like. Unsaturated hydrocarbyls contain at least one double or triple bond between adjacent carbon atoms (referred to as an “alkenyl” or “alkynyl”, respectively, if the hydrocarbyl is non-cyclic, and cycloalkeny and cycloalkynyl, respectively, if the hydrocarbyl is at least partially cyclic). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

The DSLP adjuvant may be obtained by synthetic methods known in the art, for example, the synthetic methodology disclosed in PCT International Publication No. WO 2009/035528, which is incorporated herein by reference, as well as the publications identified in WO 2009/035528, where each of those publications is also incorporated herein by reference. A chemically synthesized DSLP adjuvant, e.g., the adjuvant of formula (1), can be prepared in substantially homogeneous form, which refers to a preparation that is at least 80%, preferably at least 85%, more preferably at least 90%, more preferably at least 95% and still more preferably at least 96%, 97%, 98% or 99% pure with respect to the DSLP molecules present, e.g., the compounds of formula (1). Determination of the degree of purity of a given adjuvant preparation can be readily made by those familiar with the appropriate analytical chemistry methodologies, such as by gas chromatography, liquid chromatography, mass spectroscopy and/or nuclear magnetic resonance analysis. DSLP adjuvants obtained from natural sources are typically not easily made in a chemically pure form, and thus synthetically prepared adjuvants are preferred adjuvants of the present invention. As mentioned previously, certain of the adjuvants may be obtained commercially. A preferred adjuvant is Product No. 699800 as identified in the catalog of Avanti Polar Lipids, Alabaster Ala., see E1 in combination with E10, below.

In various embodiments of the invention, the adjuvant has the chemical structure of formula (1) but the moieties A¹, A², R¹, R², R³, R⁴, R⁵, and R⁶ are selected from subsets of the options previously provided for these moieties, where these subsets are identified below by E1, E2, etc.

E1: A1 is phosphate or phosphate salt and A2 is hydrogen.

E2: R1, R3, R5 and R6 are C3-C21 alkyl; and R2 and R4 are C5-C23 hydrocarbyl.

E3: R1, R3, R5 and R6 are C5-C17 alkyl; and R2 and R4 are C7-C19 hydrocarbyl.

E4: R1, R3, R5 and R6 are C7-C15 alkyl; and R2 and R4 are C9-C17 hydrocarbyl.

E5: R1, R3, R5 and R6 are C9-C13 alkyl; and R2 and R4 are C11-C15 hydrocarbyl.

E6: R1, R3, R5 and R6 are C9-C15 alkyl; and R2 and R4 are C11-C17 hydrocarbyl.

E7: R1, R3, R5 and R6 are C7-C13 alkyl; and R2 and R4 are C9-C15 hydrocarbyl.

E8: R1, R3, R5 and R6 are C11-C20 alkyl; and R2 and R4 are C12-C20 hydrocarbyl.

E9: R1, R3, R5 and R6 are C11 alkyl; and R2 and R4 are C13 hydrocarbyl.

E10: R1, R3, R5 and R6 are undecyl and R2 and R4 are tridecyl.

In certain options, each of E2 through E10 is combined with embodiment E1, and/or the hydrocarbyl groups of E2 through E9 are alkyl groups, preferably straight chain alkyl groups.

The DSLP adjuvant, e.g., the adjuvant of formula (1), may be formulated into a pharmaceutical composition, optionally with a co-adjuvant, each as discussed below. In this regard reference is made to U.S. Patent Publication No. 2008/0131466 which provides formulations, e.g., aqueous formulation (AF) and stable emulsion formulations (SE) for GLA adjuvant, where these formulations may be utilized for any of the DSLP adjuvants, including the adjuvants of formula (1).

The present invention provides that the DSLP adjuvant, e.g., the adjuvant of formula (1), may be utilized in combination with a second adjuvant, referred to herein as a co-adjuvant. In three embodiments of the invention, the co-adjuvant may be a delivery system, or it may be an immunopotentiator, or it may be a composition that functions as both a delivery system and an immunopotentiator, see, e.g., O'Hagan D T and Rappuoli R., Novel approaches to vaccine delivery, Pharm. Res. 21(9):1519-30 (2004). The co-adjuvant may be an immunopotentiator that operates via a member of the Toll-like receptor family biomolecules. For example, the co-adjuvant may be selected for its primary mode of action, as either a TLR4 agonist, or a TLR8 agonist or a TLR9 agonist. Alternatively, or in supplement, the co-adjuvant may be selected for its carrier properties, e.g., it may be an emulsion, a liposome, a microparticle, or alum. Optionally, two or more different adjuvants can be used simultaneously, such as by way of non-limiting example, an aluminum salt with a DSLP adjuvant, an aluminum salt with QS21, a DSLP adjuvant with QS21, and alumna aluminum salt, QS21, and MPL or GLA together. Also, Incomplete Freund's adjuvant can be used (see, e.g., Chang et al., Advanced Drug Delivery Reviews 32, 173-186 (1998)), optionally in combination with any of an aluminum salt, QS21, and MPL and all combinations thereof.

In one aspect, the co-adjuvant is alum, where this term refers to aluminum salts, such as aluminum phosphate (AlPO4) and aluminum hydroxide (Al(OH)3). When alum is used as the co-adjuvant, the alum may be present, in a dose of vaccine, in an amount of about 100 to 1,000 μg, or 200 to 800 μg, or 300 to 700 μg or 400 to 600 μg. The DSLP adjuvant, e.g., the adjuvant of formula (1), is typically present in an amount less than the amount of alum, in various aspects the DSLP adjuvant, e.g., the adjuvant of formula (1), on a weight basis, is present at 0.1-1%, or 1-5%, or 1-10%, or 1-100% relative to the weight of alum.

In one aspect, the co-adjuvant is an emulsion having vaccine adjuvanting properties. Such emulsions include oil-in-water emulsions. Freund's incomplete adjuvant (IFA) is one such adjuvant. Another suitable oil-in-water emulsion is MF-59™ adjuvant which contains squalene, polyoxyethylene sorbitan monooleate (also known as Tween™ 80 surfactant) and sorbitan trioleate. Squalene is a natural organic compound originally obtained from shark liver oil, although it is also available from plant sources (primarily vegetable oils), including amaranth seed, rice bran, wheat germ, and olives. Other suitable adjuvants are Montanide™ adjuvants (Seppic Inc., Fairfield N.J.) including Montanide™ ISA 50V which is a mineral oil-based adjuvant, Montanide™ ISA 206, and Montanide™ IMS 1312. While mineral oil may be present in the co-adjuvant, in one embodiment the oil component(s) of the vaccine compositions of the present invention are all metabolizable oils.

Examples of immunopotentiators which may be utilized in the practice of the present invention as co-adjuvants include: 3D-MPL or MPL™ adjuvant, MDP and derivatives, oligonucleotides, double-stranded RNA, alternative pathogen-associated molecular patterns (PAMPS); saponins, small-molecule immune potentiators (SMIPs), cytokines, and chemokines.

In one embodiment the co-adjuvant is 3D-MPL or MPL™ adjuvant, where the latter is commercially available from GlaxoSmithKline, although it was originally developed by Ribi ImmunoChem Research, Inc. Hamilton, Mont. See, e.g., Ulrich and Myers, Chapter 21 from Vaccine Design: The Subunit and Adjuvant Approach, Powell and Newman, eds. Plenum Press, New York (1995). Related to MPL™ adjuvant, and also suitable as co-adjuvants in the present invention, are AS02™ adjuvant and AS04™ adjuvant. AS02™ adjuvant is an oil-in-water emulsion that contains both MPL™ adjuvant and QS-21™ adjuvant (a saponin adjuvant discussed elsewhere herein). AS04™ adjuvant contains MPL™ adjuvant and alum. MPL™ adjuvant is prepared from lipopolysaccharide (LPS) of Salmonella Minnesota R595 by treating LPS with mild acid and base hydrolysis followed by purification of the modified LPS, as described more completely in the article by Ulrich and Myers.

In one embodiment, the co-adjuvant is a saponin such as those derived from the bark of the Quillaja saponaria tree species, or a modified saponin, see, e.g., U.S. Pat. Nos. 5,057,540; 5,273,965; 5,352,449; 5,443,829; and 5,560,398. The product QS-21™ adjuvant sold by Antigenics, Inc. Lexington, Mass. is an exemplary saponin-containing co-adjuvant that may be used with the DSLP adjuvant, e.g., the adjuvant of formula (1). Related to the saponins is the ISCOM™ family of adjuvants, originally developed by Iscotec (Sweden) and typically formed from saponins derived from Quillaja saponaria or synthetic analogs, cholesterol, and phospholipid, all formed into a honeycomb-like structure.

In one embodiment, the co-adjuvant is a cytokine which functions as a co-adjuvant, see, e.g., Lin R. et al. Clin. Infec. Dis. 21(6):1439-1449 (1995); Taylor, C. E., Infect. Immun. 63(9):3241-3244 (1995); and Egilmez, N. K., Chap. 14 in Vaccine Adjuvants and Delivery Systems, John Wiley & Sons, Inc. (2007). In various embodiments, the cytokine may be, e.g., granulocyte-macrophage colony-stimulating factor (GM-CSF); see, e.g., Change D. Z. et al. Hematology 9(3):207-215 (2004), Dranoff, G. Immunol. Rev. 188:147-154 (2002), and U.S. Pat. No. 5,679,356; or an interferon, such as a type I interferon, e.g., interferon-α (IFN-α) or interferon-β (IFN-β), or a type II interferon, e.g., interferon-γ (IFN-γ), see, e.g., Boehm, U. et al. Ann. Rev. Immunol. 15:749-795 (1997); and Theofilopoulos, A. N. et al. Ann. Rev. Immunol. 23:307-336 (2005); an interleukin, specifically including interleukin-1α (IL-1α), interleukin-1β (IL-1β), interleukin-2 (IL-2); see, e.g., Nelson, B. H., J. Immunol. 172(7):3983-3988 (2004); interleukin-4 (IL-4), interleukin-7 (IL-7), interleukin-12 (IL-12); see, e.g., Portielje, J. E., et al., Cancer Immunol. Immunother. 52(3): 133-144 (2003) and Trinchieri. G. Nat. Rev. Immunol. 3(2):133-146 (2003); interleukin-15 (Il-15), interleukin-18 (IL-18); fetal liver tyrosine kinase 3 ligand (Flt3L), or tumor necrosis factor (TNFα). The DSLP adjuvant, e.g., the adjuvant of formula (1), may be co-formulated with the cytokine prior to combination with the vaccine antigen, or the antigen, DSLP adjuvant, e.g., the adjuvant of formula (1) and cytokine co-adjuvant may be formulated separately and then combined.

In one embodiment, the co-adjuvant is unmethylated CpG dinucleotides, optionally conjugated to the flu antigen described herein.

In one embodiment, the co-adjuvant is a cyclic di-nucleotide. Exemplary cyclic di-nucleotides are the bacterial second messangers, c-di-GMP, c-di-AMP, and c-di-IMP (see, e.g. McWhirter et al, J Exp. Med. 206:1899-1911, 2009; Woodward et al, Science 328:1703-1705, 2010; Ebenson et al, Clin. Vaccine Immunol 14:952-958, 2007).

When a co-adjuvant is utilized in combination with the DSLP adjuvant, e.g., the adjuvant of formula (1), the relative amounts of the two adjuvants may be selected to achieve the desired performance properties for the vaccine composition which contains the adjuvants, relative to the antigen alone. For example, the adjuvant combination may be selected to enhance the antibody response of the antigen, and/or to enhance the subject's innate immune system response. Activating the innate immune system results in the production of chemokines and cytokines, which in turn activate an adaptive (acquired) immune response. An important consequence of activating the adaptive immune response is the formation of memory immune cells so that when the host re-encounters the antigen, the immune response occurs quicker and generally with better quality.

4. Formulation

Immunogenic compositions comprise one or more SLPs and a DSLP adjuvant, e.g., an adjuvant of formula (1), such as GLA. The compositions may comprise SLPs from multiple proteins. The composition may also contain co-adjuvants such as aluminum salts (e.g., alum), excipients, carriers, buffers, stabilizers, binders, preservatives such as thimerosal, surfactants, etc. as known in the art and discussed below.

In one aspect, the adjuvant and the SLPs are prepared as separate solutions (suspension) and then those solutions are combined in order to provide the immunogenic composition. In preparing the DSLP adjuvant composition, the DSLP adjuvant may be formulated in various ways, e.g., as an oil-in-water emulsion, as a water-in-oil emulsion, as an aqueous solution or suspension, or in a liposome or niosome.

The DSLP adjuvant may be formulated in a completely oil-free form or it may be present in a composition that contains less than about 1% v/v oil. In order to prepare an oil-free composition, water, adjuvant (e.g., GLA is a preferred adjuvant) and a surfactant, e.g., a phospholipid, e.g., 1-palmitoyl-2-oleoyl-sn-glycero-3-phosphocholine (POPC) may be combined. The composition may be prepared by adding a solution of ethanol and POPC to a pre-weighed amount of GLA. This wetted GLA is sonicated for 10 minutes to disperse the GLA as much as possible. The GLA is then dried under nitrogen gas. The dried GLA and POPC are reconstituted with WFI (water-for-injection) to the correct volume. This solution is sonicated at 60° C. for 15-30 minutes until all the GLA and POPC are in solution. For long term storage, GLA-AF formulations must be lyophilized. The lyophilization process consists of adding glycerol to the solution until it is 2% of the total volume. Then the solution is placed in vials in 1-10 mL amounts. The vials are run through a lyophilization process which consists of freezing the solution and then putting it under vacuum to draw off the frozen water by sublimation.

When the adjuvant will be combined with an oil, then for use in humans, the oil is preferably metabolizable. When an oil is present, then it is preferable to also have an anti-oxidant present in the composition. The oil may be any vegetable oil, fish oil, animal oil or synthetic oil; the oil should not be toxic to the recipient and is capable of being transformed by metabolism. Nuts (such as peanut oil), seeds, and grains are common sources of vegetable oils. Particularly suitable metabolizable oils include squalene (2,6,10,15,19,23-hexamethyl-2,6,10,14,18,22-tetracosahexane), an unsaturated oil found in many different oils, and in high quantities in shark-liver oil. Squalene is an intermediate in the biosynthesis of cholesterol. The average size of the oil droplets is typically less than 1 micron, may be in the range of 30-600 nm, and usually about 80 to about 120 nm or less than about 150 nm. Oil droplet size may be measured by photon correlation spectroscopy. Typically, at least about 80% of the oil droplets should be within the desired ranges, or at least about 90% or at least about 95%. The fraction of oil in the emulsions is generally in the range of 2 to 10% (e.g., about 2%, about 3%, about 4%, about 5%, about 6%, about 7%, about 8%, about 9% and about 10%);

The composition, particularly an oil-in-water emulsion, may contain an anti-oxidant, such as alpha-tocopherol (vitamin E, U.S. Pat. No. 5,650,155, U.S. Pat. No. 6,623,739). The amount of an anti-oxidant, such as alpha-tocopherol, is preferably from about 2 to about 10%. Preferably the ratio of oil:alpha tocopherol is equal or less than 1 as this provides a more stable emulsion.

In some cases it may be advantageous that the vaccines of the present invention will further contain additional components. For example, sorbitan trioleate (e.g., Span® 85) may also be present at a level of about 1%. Stabilizers, such as a triglyceride, ingredients that confer isotonicity, and other ingredients may be added. When present, a surfactant may be included at a concentration from about 0.3 to 3%. In addition to the DSLP adjuvant, the adjuvant-containing compositions may also comprise buffers, stabilizers, excipients, preservatives, carriers, or other non-active ingredients. Additives are typically pharmaceutically acceptable and bio-compatible. Additional adjuvants may be present, as described in more detail elsewhere herein. These co-adjuvants include, 2′-5′ oligo A, bacterial endotoxins, RNA duplexes, single stranded RNA, lipoprotein, peptidoglycan, flagellin, CpG DNA, lipopolysaccharide, MPA (monophosphoryl lipid A), 3-O-deacylated MPL, lipopolysaccharide, QS21 (a saponin), aluminium hydroxide (“alum”) and other mineral salts, oil emulsions (e.g., MF59™, R848 and other imidazoquinolines, virosomes and other particulate adjuvants (see, Vogel and Powell, “A compendium of vaccine adjuvants and excipients” Pharm Biotechnol 6:141-228, 1995; incorporated in its entirety).

The method of producing oil in water emulsions is well known to the person skilled in the art. Commonly, the method comprises mixing the oil phase with a surfactant, such as phosphatidylcholine, block co-polymer, or a TWEEN80® solution, followed by homogenization using a homogenizer. For instance, a method that comprises passing the mixture once, twice or more times through a syringe needle would be suitable for homogenizing small volumes of liquid. Equally, the emulsification process in a microfluidiser (M110S microfluidics machine, maximum of 50 passes, for a period of 2 min at maximum pressure input of 6 bar (output pressure of about 850 bar)) could be adapted to produce smaller or larger volumes of emulsion. This adaptation could be achieved by routine experimentation comprising the measurement of the resultant emulsion until a preparation was achieved with oil droplets of the required diameter. Other equipment or parameters to generate an emulsion may also be used.

An exemplary oil-in-water emulsion using squalene is known as “SE” and comprises squalene, glycerol, phosphatidylcholine or lecithin or other block co-polymer as a surfactant in an ammonium phosphate buffer pH 5.1 with alpha-toceraphol. When GLA is used as the DSLP, the resulting composition is referred to herein as GLA-SE. To make such a composition, GLA (100 micrograms; Avanti Polar Lipids, Inc., Alabaster, Ala.; product number 699800) is emulsified in squalene (34.3 mg) with glycerol (22.7 mg), phosphotidylcholine or lecithin (7.64 mg), Pluronic® F-68 (BASF Corp., Mount Olive, N.J.) or similar block co-polymer (0.364 mg) in 25 millimolar ammonium phosphate buffer (pH=5.1) optionally using 0.5 mg D,L-alpha-tocopherol as an antioxidant. The mixture is processed under high pressure until an emulsion forms that does not separate and that has an average particle size of less than 180 nm. The emulsion is then sterile-filtered into glass unidose vials and capped for longer term storage. This preparation may be used for at least three years when stored at 2-8° C. Other oil-containing compositions that include a DSLP and a protein as described herein may be prepared in analogy to those compositions prepared as described in U.S. Pat. Nos. 5,650,155; 5,667,784; 5,718,904; 5,961,970; 5,976,538; 6,630,161; and 6,572,861.

Some particular compositions and vaccines comprise SLPs for NY-ESO-1, SLPs for TRP2, SLPs for CAIX, SLPs for MAGE A3, and SLPs for UL19 of HSV-2 and SIV-Gag as in the Examples below. Typically the SLPs are formulated with GLA, either with or without SE.

In various embodiments, the amount of SLP in an immunogenic composition is between about 0.001 μg to about 5000 μg of total SLP, about 0.01 to about 1000 μg of total SLP, about 0.01 to about 100 μg of total SLP, about 0.01 to about 50 μg of total SLP, about 0.05 to about 100 μg of total SLP, about 0.1 to about 100 μg of total SLP, about 0.5 to about 100 μg of total SLP, about 0.1 to about 50 μg of total SLP, about 0.1 to about 25 μg of total SLP, or about 0.5 to about 25 μg of total SLP. The amount of SLP in an immunogenic composition is typically a low dose e.g., from about 0.01 μg to about 100 μg of total SLP in a single dose, or about 0.1 μg to about 15 μg of total SLP in a single dose. The low amount may be any of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 μg of total SLP. Any of these amounts may be combined with any other amount to form all possible ranges without having to set forth each combination herein. The aforementioned dose ranges and amounts reflect the contemplated values for an SLP of approximately 45-50 amino acids in length. For peptides of larger MW (e.g., 50-60 amino acids in length), contemplated dose ranges include, for example, 0.0150 μg to about 150 μg of total SLP in a single dose, or about 0.15 μg to about 50 μg of total SLP in a single dose. For peptides of lower MW (e.g., 30-40 amino acids in length), contemplated dose ranges include, for example, 0.005 μg to about 75 μg of total SLP in a single dose, or about 0.05 μg to about 10 μg of total SLP in a single dose.

As described herein, an optimum dosage for an SLP comprising a CD4 epitope may be different than the optimum dosage for an SLP comprising a CD8 eptitope (e.g., the dosages required to elicit a strong CD4 response and a strong CD8 response may differ). By way of example, the amount of SLP comprising a CD4 epitope in an immunogenic composition is between about 0.01 to about 100 μg of total SLP, about 0.1 to about 75 μg of total SLP, about 1.0 to about 15 μg of total SLP, and the amount of SLP comprising a CD8 epitope in an immunogenic composition is between about 1.0 to about 500 μg of total SLP, about 5 to about 100 μg of total SLP, or about 10 to about 75 μg of total SLP.

The optimal low dose of SLP will be that which demonstrates greater immunogenicity relative to that demonstrated by higher doses of SLP in identical formulations, e.g. formulated with the same dose of adjuvant and/or co-adjuvant and carrier. The low amount of SLP protein may be as low as practically feasible provided that it allows formulation of a vaccine that meets an international (e.g. EU or FDA) criterion for efficacy, as detailed below. The dose amount will typically be determined by immunogenicity, number of intended administrations, and the size and condition of the subject. As described in the Examples herein, when comparing the efficacy of a composition comprising a SLP versus a full-length protein, the amount of SLP and full length protein is preferably reported in per mole or molar terms in order to make comparisons of vaccine potency that are based on the amounts of the amino acid sequences that make up the T-cell determinants within the immunogens. In various embodiments of the instant disclosure, a greater immune response is observed per mole of SLP epitope versus full-length protein.

The SLPs may be packaged as a solution, but can also be packaged in dry form (e.g., desiccated), in which case, a user adds any necessary liquid. Typically, additives such as buffers, stabilizers, preservatives, excipients, carriers, and other non-active ingredients will also be present in the package. Additives are typically pharmaceutically acceptable and bio-compatible.

The amount DSLP adjuvant, e.g. the adjuvant of formula (1), e.g., GLA, that is used in a dose of composition of the present invention (where a dose is an amount of composition administered to the subject in need thereof) that also contains antigen, useful as a vaccine, is in one embodiment about 0.5 μg to about 50 μg, in another embodiment is about 1.0 μg to 25 μg, and in various other embodiments of the present invention may be about 1 μg, about 2 μg, about 2.5 μg, about 5 μg, about 7.5 μg, about 10 μg, about 15 μg, about 20 μg or about 25 μg. The total volume of composition in a dose will typically range from 0.5 mL to 1.0 mL. An emulsion, such as SE, may be present in the composition, where the oil component(s) of the emulsion constitutes, in various embodiments, at about 0.1%, about 0.5%, about 1.0%, about 1.5%, about 2%, about 2.5%, about 3%, about 4%, about 5%, about 7.5% or about 10% of the total volume of the composition.

DSLP adjuvant, e.g., the adjuvant of formula (1), and protein may be provided in separate containers and mixed on-site or pre-mixed. In addition, the protein may be presented in separate containers or combined in a single container. A container can be a vial, ampoule, tube, or well of a multi-well device, reservoir, syringe or any other kind of container. The container or containers may be provided as a kit. If one or more of the containers comprises desiccated ingredients the liquids for reconstitution may be provided in the kit as well or provided by the user. The amount of solution in each container or that is added to each container is commensurate with the route of administration and how many doses are in each container. A vaccine given by injection is typically from about 0.1 ml to about 1.0 ml, while a vaccine that is given orally may be a larger volume, from about 1 ml to about 10 ml for example. Suitable volumes may also vary according to the size and age of the subject.

The compositions are generally provided sterile. Typical sterilization methods include filtration, irradiation and gas treatment.

5. Administration

The immunogenic composition can be administered by any suitable delivery route, such as intradermal, mucosal (e.g., intranasal, oral), intramuscular, subcutaneous, sublingual, rectal, vaginal. Other delivery routes are well known in the art.

The intramuscular route is one suitable route for the vaccine composition. Suitable i.m. delivery devices include a needle and syringe, a needle-free injection device (for example Biojector, Bioject, Oreg. USA), or a pen-injector device, such as those used in self-injections at home to deliver insulin or epinephrine. Intradermal and subcutaneous delivery are other suitable routes. Suitable devices include a syringe and needle, syringe with a short needle, and jet injection devices.

The SLP vaccine may be administered by a mucosal route, e.g., intranasally. Many intranasal delivery devices are available and well known in the art. Spray devices are one such device. Oral administration is as simple as providing a solution for the subject to swallow.

SLP vaccine may be administered at a single site or at multiple sites. If at multiple sites, the route of administration may be the same at each site, e.g., injection in different muscles, or may be different, e.g., injection in a muscle and intranasal spray. Furthermore, the vaccine may be administered at a single time point or multiple time points. Generally if administered at multiple time points, the time between doses has been determined to improve the immune response.

SLP vaccine is administered at a dose sufficient to effect a beneficial immune response to prevent or lessen symptoms of disease or infection. One preferred indicator of a beneficial response is an increased amount or function or frequency of CD8 or CD4 T-cells responsive to the SLPs.

Assays for T-cell function include IFN-γ ELISPOT and ICS (intracellular cytokine staining). By measuring cytokine production for several cytokines Th1/Th2 profiles can be established. In particular, a desirable pattern is increases in IFN-γ and IL-2 production and reduced IL-5 and IL-4 production. ELISPOT assay detecting interferon-gamma is widely used to quantize CD4 and CD8 T-cell responses to candidate vaccines. The ELISPOT assay is based on the principle of the ELISA detecting antigen-induced secretion of cytokines trapped by an immobilized antibody and visualized by an enzyme-coupled second antibody. ICS is a routinely used method to quantify cytotoxic T-cells by virtue of cytokine expression following stimulation with agonists, such as antibodies to T-cell surface molecules or peptides that bind MHC Class molecules. Exemplary procedures of ICS and ELISPOT are described in the examples below.

Antigen-specific T-cell function can also be measured. Influenza-specificspecific-CD4+T-cells that co-express IFNγ, IL-2 and TNF have better functional activity and costimulatory potential relative to cells that produce a single cytokine. Thus, the induction of multiple cytokine-producing CD4+ T-cells is desirable. Antigen-specific T-cell stimulation assays may be used to estimate the frequency of CD4 T-cells that produce IFNγ, IL-2, TNFα, and combinations thereof by flow cytometry. The addition of IL-5 to this assay can be used to distinguish Th1 vs Th2 CD4+ cells. A time course experiment at 3, 6, 12, and 24 weeks post-immunization is performed to determine long-lasting T-cell responses. Flow cytometry can also be used to measure and distinguish the generation of effector memory CD4+ T-cells (TEM: CD4+CD62L-CCR7-IFNγ+) and central memory CD4 T (TCM:CD4+CD62L+CCR7+IL2+IFNγ+/−) cells. Vaccine formulations that induce production of IFNγ, TNF and IL-2 and increase CD4CM are desirable. Cytotoxic CD8+ T-cells also play a role in clearing virus load and limiting disease progression. Vaccines that elicit antigen-specific CD8+ T-cells are desirable.

The compositions may be administered as a single dose (e.g., injection) or as multiple doses. When multiple doses are administered, generally the second and subsequent doses are administered after an interval of time. Often administration of the initial dose is called “priming” the immune response, and administration of subsequent doses are called “boosting” the immune response. Typically, the time between the first and second administration is at least 2 weeks, although shorter or longer time periods may be used. Additional doses may be administered at least 2-4 weeks following the earlier administration, and in some cases, may be administered long (e.g., 1 year) after the earlier dose. Administration of dose(s) following infection or onset of a disease serves to treat the infection or disease.

Methods are provided herein that comprise administering the SLP compositions of the present invention with at least one different immunogenic composition comprising the antigen from which the SLPs are derived for inducing an adaptive, antigen-specific immune response against the immunogenic target. Dual immunization of a subject with the compositions as described herein results in induction of a humoral immune response and a cellular immune response (including a CD4 T-cell response and a CD8 T-cell response). Accordingly, provided herein are methods for inducing a humoral immune response and a cellular response, which comprises a CD4 T-cell response and/or a CD8 T-cell response (and which may include a cytotoxic T-cell response), wherein each of the immune responses is specific for an immunogen(s) and thereby specific for the respective designated antigen(s). These methods comprise administering an immunogenic composition that comprises SLPs and a second immunogenic composition comprising at least one immunogen, which is derived from the same antigen target at the SLPs. The immunogenic antigen is isolated and/or recombinantly produced. The immunogenic antigen may be in the form of a viral-like particle. The immunogenic antigen may comprise amino acid sequences that include two different immunogenic regions or epitopes of a designated antigen of interest. The immunogenic antigen may comprise at least one B cell epitope or may comprise a T-cell epitope or may comprise amino acid sequences that include both a B cell epitope and a T-cell epitope. The two immunogenic compositions may be administered concurrently or sequentially in either order. In another embodiment, the SLP composition may be administered as a prime and the immunogenic antigen composition administered as a boost.

The immunogenic composition that comprises at least one immunogenic antigen may further comprise at least one adjuvant that is pharmaceutically or physiologically suitable for administering to the subject in need thereof to whom the immunogenic compositions are administered. If both the composition comprising SLPs and the composition comprising an immunogenic antigen comprise an adjuvant, the adjuvants may be the same or different. Immunogens and adjuvants are discussed in detail herein.

The compositions may be administered along with other agents, such as medicines, drugs, herbs, etc. Other agents include those that treat symptoms, such as cough syrup, aspirin, NSAIDs such as ibuprofen may also be provided.

The pharmaceutical compositions, SLP vaccine compositions, and kits described herein may be administered to individuals to prevent, to protect against, or to treat infections and diseases, including cancer.

### Exemplary Embodiments

In some embodiments of the disclosure, an immunogenic fragment of UL19 (SEQ ID NO: 1) up to 60, up to 55, up to 50, up to 45, up to 40, up to 35 or up to 30 amino acids in length that comprises any of SEQ ID NOS: 3-12 or SEQ ID NOS: 13-17 is provided. In various other embodiments, an immunogenic fragment of SW-Gag (SEQ ID NO: 2) up to 60, up to 55, up to 50, up to 45, up to 40, up to 35 or up to 30 amino acids in length that comprises any of SEQ ID NOS: 18-20 is provided. A polypeptide comprising any of the preceding immunogenic fragments is also provided according to the present disclosure.

In various other embodiments, a composition or vaccine comprising any of the preceding immunogenic fragments (e.g., a viral antigen such as UL19, or a cancer antigen such as NY-ESO-1 or MAGE A3) and an adjuvant, optionally with a sterile pharmaceutically acceptable carrier, is provided. In various embodiments, the adjuvant is a TLR4 agonist. Exemplary adjuvants and co-adjuvants are described herein.

In various embodiments of the disclosure, a composition or vaccine comprising (a) any one of the preceding immunogenic fragments comprising a CD4 epitope, and (b) another of the preceding immunogenic fragments comprising a CD8 epitope, and (c) an adjuvant, optionally with a sterile pharmaceutically acceptable carrier, is provided. By way of example, a composition or vaccine comprising (a) any one of the preceding immunogenic fragments of UL19 comprising a CD4 epitope (any of SEQ ID NOS: 3-7), and (b) another of the preceding immunogenic fragments of UL19 comprising a CD8 epitope (any of SEQ ID NOS: 8-12), (c) an adjuvant, optionally with a sterile pharmaceutically acceptable carrier, is provided. In another example, a composition or vaccine comprising (a) any one of the preceding immunogenic fragments of SIV-Gag comprising a CD4 epitope (e.g., SEQ ID NO: 19 and (b) another of the preceding immunogenic fragments of SIV-Gag comprising a CD8 epitope (e.g., SEQ ID NO: 20 (c) an adjuvant, optionally with a sterile pharmaceutically acceptable carrier, is provided.

In various embodiments, the use of any of these compositions for inducing an immune response, optionally via intradermal or subcutaneous administration, is provided.

In one embodiment, the adjuvant is a TLR4 agonist. In other embodiments, the adjuvant is a monophosphoryl lipid A, preferably a monophosphoryl lipid A that is a TLR4 agonist, e.g., GLA.

In addition to any of the foregoing embodiments described in the detailed description, embodiments are contemplated including any of the following or any combinations thereof:

1. An immunogenic composition comprising one or more SLPs (synthetic long peptides) and a TLR4 agonist adjuvant, wherein the one or more SLPs:

a) are up to 60, up to 55, up to 50, up to 45, up to 40, up to 35 or up to 30 amino acids in length; and

b) comprise at least one CD4 T-cell epitope and/or at least one CD8 T-cell epitope;

wherein the one or more SLPs are present in the composition in an amount to generate a CD4 T-cell and/or CD8 T-cell response after one, two, three, four or five administrations.

2. The composition according to embodiment 1 wherein the at least one CD4 T-cell epitope and at least one CD8 T-cell epitope are present in an amount to generate a CD4 T-cell and a CD8 T-cell response, respectively.

3. The composition according to embodiment 1 wherein only the at least one CD4 T-cell epitope in an amount to generate a CD4 T-cell is present.

4. The composition according to embodiment 1 wherein only the at least one CD8 T-cell epitope in an amount to generate a CD8 T-cell is present.

5. The composition according to embodiment 1 wherein the adjuvant is GLA.

6. The composition according to any one of embodiments 1-2 and 4-5 wherein the one or more SLPs comprising at least one CD8 epitope is administered at higher concentration compared to the one or more SLPs comprising at least one CD4 epitope.

7. The composition according to embodiment 6 wherein the amount of the one or more SLPs comprising at least one CD8 epitope in the composition is between about 1.0 to about 500 μg of total SLP.

8. The composition according to embodiment 7 wherein the amount of the one or more SLPs comprising at least one CD8 epitope in the composition is between about 5 to about 100 μg of total SLP.

9. The composition according to embodiment 6 wherein the amount of the one or more SLPs comprising at least one CD4 epitope in the composition is between about 0.01 to about 100 μg of total SLP.

10. The composition according to embodiment 9 wherein the amount of the one or more SLPs comprising at least one CD4 epitope in the composition is between about 0.1 to about 75 μg of total SLP.

11. The composition according to any one of embodiments 1-10 wherein the one or more SLPs are derived from an HSV-2 protein.

12. The composition according to embodiment 11 wherein the HSV-2 protein is UL19.

13. The composition according to any one of embodiments 1-10 wherein the one or more SLPs are derived from a virus-derived antigen selected from the group the group consisting of an HIV antigen, an SIV antigen, an adenovirus antigen, an enterovirus antigen, a coronavirus antigen, a calicivirus antigen, a distemper virus antigen, an Ebola virus antigen, a flavivirus antigen, a hepatitis virus antigen, a herpesvirus antigen, an infectious peritonitis virus antigen, an influenza virus antigen, a leukemia virus antigen, a Marburg virus antigen, an orthomyxovirus antigen, a papilloma virus antigen, a parainfluenza virus antigen, a paramyxovirus antigen, a parvovirus antigen, a pestivirus antigen, a picorna virus antigen, a poliovirus antigen, a pox virus antigen, a rabies virus antigen, a reovirus antigen, a retrovirus antigen, or a rotavirus antigen.

14. The composition according to any one of embodiments 1-10 wherein the one or more SLPs are derived from a cancer antigen.

15. The composition according to embodiment 14 wherein the cancer antigen is carbonic anhydrase IX, NY-ESO-1, MAGE, BAGE, RAGE, MART-1/Melan-A, gp100, gp75, mda-7, tyrosinase, tyrosinase-related protein, 5T4, SM22-alpha, carbonic anhydrase I, HIF-1alpha, HIF-2alpha, PSMA, PSA, STEAP, and NKX3.1.

In addition to any of the foregoing embodiments, embodiments are contemplated including any of the following methods or any combinations thereof:

16. A method of inducing an immune response in a subject against an antigen comprising administering an immunogenic composition according to any one of embodiments 1-15.

17. The method according to embodiment 16 comprising:

a) at least one first administration (prime) of a composition comprising one or more SLPs comprising at least one CD4 epitope; and

b) at least one second administration (boost) of a composition comprising one or more SLPs comprising at least one CD8 epitope.

18. The method according to embodiment 16 comprising:

a) at least one first administration (prime) of a composition comprising one or more SLPs comprising at least one CD8 epitope; and

b) at least one second administration (boost) of a composition comprising one or more SLPs comprising at least one CD4 epitope.

19. The method according to any one of embodiments 17-18 wherein the amount of the one or more SLPs comprising at least one CD8 epitope in the composition is between about 1.0 to about 500 μg of total SLP.

20. The method according to embodiment 19 wherein the amount of the one or more SLPs comprising at least one CD8 epitope in the composition is between about 5 to about 100 μg of total SLP.

21. The method according to any one of embodiments 17-18 wherein the amount of the one or more SLPs comprising at least one CD4 epitope in the composition is between about 0.01 to about 100 μg of total SLP.

22. The method according to embodiment 21 wherein the amount of the one or more SLPs comprising at least one CD4 epitope in the composition is between about 0.1 to about 75 μg of total SLP.

23. The methods according to any one of embodiments 17-22 wherein the composition comprising the one or more SLPs comprising at least one CD4 epitope and the composition comprising the one or more SLPs comprising at least one CD8 epitope are administered at different sites.

24. The methods according to any one of embodiments 17-23 wherein the composition comprising the one or more SLPs comprising at least one CD4 epitope and the composition comprising the one or more SLPs comprising at least one CD8 epitope are administered at different times.

25. The method according to embodiment 16 wherein the immunogenic composition comprises both an SLP comprising at least one CD4 T-cell epitope and an SLP comprising at least one CD8 T-cell epitope.

26. The method according to embodiment 16 wherein the immunogenic composition comprises at least one SLP comprising a CD4 T-cell epitope and a CD8 T-cell epitope.

27. The method according to any one of embodiments 16-18 wherein the SLP comprising at least one CD4 T-cell epitope is present in a greater amount than the SLP comprising at least one CD8 T-cell epitope.

28. The method according to embodiment 17 further comprising a second administration (prime) of a composition comprising one or more SLPs comprising at least one CD4 epitope prior to a third administration (boost) of a composition comprising one or more SLPs comprising at least one CD8 epitope.

29. The method according to embodiment 18 further comprising a second administration (prime) of a composition comprising one or more SLPs comprising at least one CD8 epitope prior to a third administration (boost) of a composition comprising one or more SLPs comprising at least one CD4 epitope.

30. The method of embodiment 16, wherein the adjuvant is GLA and the antigen is UL19 or a virus-derived antigen selected from the group consisting of HIV antigen, an SIV antigen, an adenovirus antigen, an enterovirus antigen, a coronavirus antigen, a calicivirus antigen, a distemper virus antigen, an Ebola virus antigen, a flavivirus antigen, a hepatitis virus antigen, a herpesvirus antigen, an infectious peritonitis virus antigen, an influenza virus antigen, a leukemia virus antigen, a Marburg virus antigen, an orthomyxovirus antigen, a papilloma virus antigen, a parainfluenza virus antigen, a paramyxovirus antigen, a parvovirus antigen, a pestivirus antigen, a picorna virus antigen, a poliovirus antigen, a pox virus antigen, a rabies virus antigen, a reovirus antigen, a retrovirus antigen, or a rotavirus antigen

31. The method of embodiment 16, wherein the adjuvant is GLA and the antigen is carbonic anhydrase IX, NY-ESO-1, MAGE, BAGE, RAGE, MART-1/Melan-A, gp100, gp75, mda-7, tyrosinase, tyrosinase-related protein, 5T4, SM22-alpha, carbonic anhydrase I, HIF-1alpha, HIF-2alpha, PSMA, PSA, STEAP, and NKX3.1.

32. The method accordin to any one of embodiments 16-31 wherein the composition is administered subcutaneously.

33. The method accordin to any one of embodiments 16-31 wherein the composition is administered intramuscularly.

In addition to any of the foregoing embodiments, embodiments are contemplated including any of the following any combinations thereof:

34. Any of the preceding embodiments, wherein the amounts administered are effective to induce a cytotoxic T lymphocyte response against a cell bearing the antigen, e.g. against a tumor cell or against a microorganism. In any of the preceding embodiments, the amounts administered are effective to reduce the likelihood of occurrence or recurrence of a tumor comprising the tumor-associated antigen. In any of the preceding embodiments, the amounts administered are effective to reduce the likelihood of occurrence or severity of a disease caused by the microorganism. Such methods can prevent or treat an infection caused by the infectious microorganism.

35. Any of the preceding embodiments wherein the TLR4 agonist adjuvant or non-toxic lipid A-related adjuvant, is a monophosphoryl lipid A, or 3 De-O-acylated monophosphoryl lipid A (MPL), or a lipid A mimetic, or GLA of formula I as described in its entirety above, or GLA of formula (Ia):

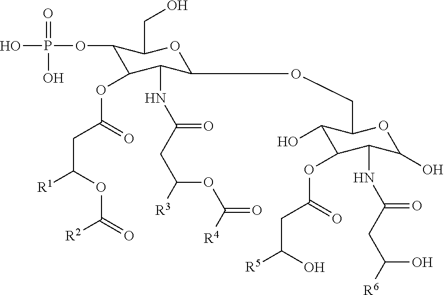

or a pharmaceutically acceptable salt thereof, where: R1, R3, R5 and R6 are C11-C20 alkyl; and R2 and R4 are C12-C20 alkyl; in a more specific embodiment, the GLA has the formula (Ia) set forth above wherein R1, R3, R5 and R6 are C11-14 alkyl; and R2 and R4 are C12-15 alkyl; in a further more specific embodiment, the GLA has the formula (Ia) set forth above wherein R1, R3, R5 and R6 are C11 alkyl, e.g., undecyl; and R2 and R4 are C13 alkyl, e.g., tridecyl;

or the GLA has a structure selected from the following chemical formula (Ib):

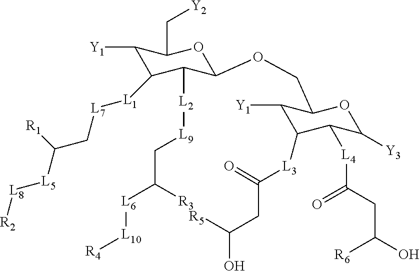

or a pharmaceutically acceptable salt thereof, wherein: L1, L2, L3, L4, L5 and L6 are the same or different and are independently selected from O, NH, and (CH2); L7, L8, L9 and L10 are the same or different, and at any occurrence may be either absent or C(═O); Y1 is an acid functional group; Y2 and Y3 are the same or different and are each independently selected from OH, SH, and an acid functional group; Y4 is OH or SH; R1, R3, R5 and R6 are the same or different and are each independently selected from the group of C8-C13 alkyl; and R2 and R4 are the same or different and are each independently selected from the group of C6-C11 alkyl.

The following examples are offered by way of illustration, and not by way of limitation.

### EXAMPLE 1

### Vaccination with SLPs+GLA-SE is Effective at Generating Antigen-Specific CD4 And CD8 T-Cell Responses

This Example addresses the questions of whether (1) CD4 and CD8 T-cell responses are generated against known determinants after immunization with SLP+GLA-SE, and (2) if responses are observed, how such responses compare to immunization with full-length recombinant protein+GLA-SE.

A set of ten T-cell epitopes (CD4 or CD8) from UL19 was identified as being of interest for inclusion in a vaccine composition. Those epitopes are set forth in Table A as SEQ ID NOs 3-12.

TABLE A

| SEQ | | | |
|---|---|---|---|
| ID | EPITOPE | LOCATION | EPITOPE |
| NO: | SEQUENCE | IN UL19 | TYPE |
| 3 | IQNGEYFYVLPVHAL | aa 949-963 | CD4 |
| 4 | EYFYVLPVHALFAGA | aa 953-967 | CD4 |
| 5 | NYFSSIRQPVVQHAR | aa 997-1,001 | CD4 |
| 6 | SIRQPVVQHARESAA | aa 1,001-1,015 | CD4 |
| 7 | CEFIATPVATDINYF | aa 1,185-1,199 | CD4 |
| 8 | LGLSVACVCTKFPEL | aa 73-87 | CD8 |
| 9 | VACVCTKFPELAYMN | aa 77-91 | CD8 |
| 10 | SAAGENALTYALMAG | aa 1,013-1,027 | CD8 |
| 11 | ENALTYALMAGYFKM | aa 1,017-1,031 | CD8 |
| 12 | HPGFGFTVVRQDRFV | aa 1,045-1,059 | CD8 |

In order to prepare a vaccine composition containing the epitopes of Table A, a set of 5 SLPs was synthesized. Those 5 SLPs are described in Table B. Each SLP contains an amino acid sequence which is found in UL19. The second column of Table B indicates where, in UL19, the SLP amino acid sequence can be found. The third column of Table B provides the SLP sequence and, with underlining and italication, delineates the epitope(s) present in the SLP. The fourth column identifies the type(s) of epitope(s) present in the corresponding SLP, and relates that to the SEQ ID NO. from Table A. The vendor who prepared the SLPs for this study was AnaSpec, Inc. (Fremont, Calif., USA).

TABLE B

| SEQ | REGION OF | | | Epitope |
|---|---|---|---|---|
| ID | UL19 | | | containing15- |
| NO. | PROTEIN | SEQUENCE | EPITOPE | mer |
| 13 | aa 1,171-1,215 | VPRRAGMDHGQDA | CD4 (SEQ ID NO: 7 | 297 |
| | | VCEFIATPVATDINY | underlined) | |
| | | FRRPCNPRGRAAGG | | |
| | | VYA | | |
| 14 | aa 936-994 | GVLLMAPQHLDHTI | CD4 (SEQ ID NO: 3 | 238 & 239 |
| | | QNGEYFYVLPVHA | underlined; SEQ ID | |
| | | LFAGADHVANAPN | NO: 4 bolded) | |
| | | FPPAL | | |
| 15 | aa 1,030-1,074 | KMSPVALYHQLKTG | CD8 (SEQ ID NO: | 262 |
| | | LHPGFGFTVVRQDR | 12 underlined) | |
| | | FVTENVLFSERASEA | | |
| | | YF | | |
| 16 | aa 48-104 | SYCNTLSLVRFLELG | CD8 (SEQ ID NO: 8 | 19 & 20 |
| | | LSVACVCTKFPELA | underlined; SEQ ID | |
| | | YMNEGRVQFEVHQ | NO: 9 bolded) | |
| | | PLI | | |
| 17 | aa 992-1,206 | PALGANYFSSIRQPV | CD4 and CD8 (SEQ | 250, 251, |
| | | VQHARESAAGENAL | ID NO: 5 underlined; | 254, 255 |
| | | TYALMAGYFKMSP | SEQ ID NO: 6 | |
| | | VAL | italicized; SEQ ID | |
| | | | NO: 10 bold; SEQ ID | |
| | | | NO: 11 double | |
| | | | underline) | |

Various doses of SLPs or recombinant full length UL19 protein were formulated with GLA-SE as shown in Table C. A mixture comprising equal weights of each of the five SLPs identified in Table B in DMSO was combined with 5 μg GLA in 2% SE (i.e., 2 wt % oil in an oil-in-water emulsion). Samples were brought to the final volumes for injection in PBS. The amount of antigen in a 50 μL dose is shown in Table C.

TABLE C

| | | | Dose relationship to |
|---|---|---|---|
| | Total antigen | Dose each SLP | aa sequence in |
| Antigen | dose | (mass) | native rP (molar) |
| rUL19 protein | 5 μg | N/A | N/A |
| 5 UL19 SLPs | 180 μg | 36 μg | 216X |
| 5 UL19 SLPs | 30 μg | 6 μg | 36X |
| 5 UL19 SLPs | 5 μg | 1 μg | 6X |
| 5 UL19 SLPs | 0.825 μg | 0.165 μg | equimolar |

Groups of five B6 mice were immunized with 50 μL of the antigen formulations identified in Table C by injection either intramuscularly (i.m.) in one hind limb or subcutaneously (s.c) at the base of the tail. On day 21 post-immunization, mice were administered a second dose of vaccine (boost) identical to the first dose (prime). Antigen-specific splenic CD4 and CD8 T-cell responses were measured on day 5 post-boost. Intracellular cytokine staining (ICS) for IFN-γ, TNF-α, and IL-2 was performed after ex-vivo re-stimulation of splenocyte cultures for 5 hours with UL19 peptides SEQ ID NOs: 4, 5, 7, 9, and 12.

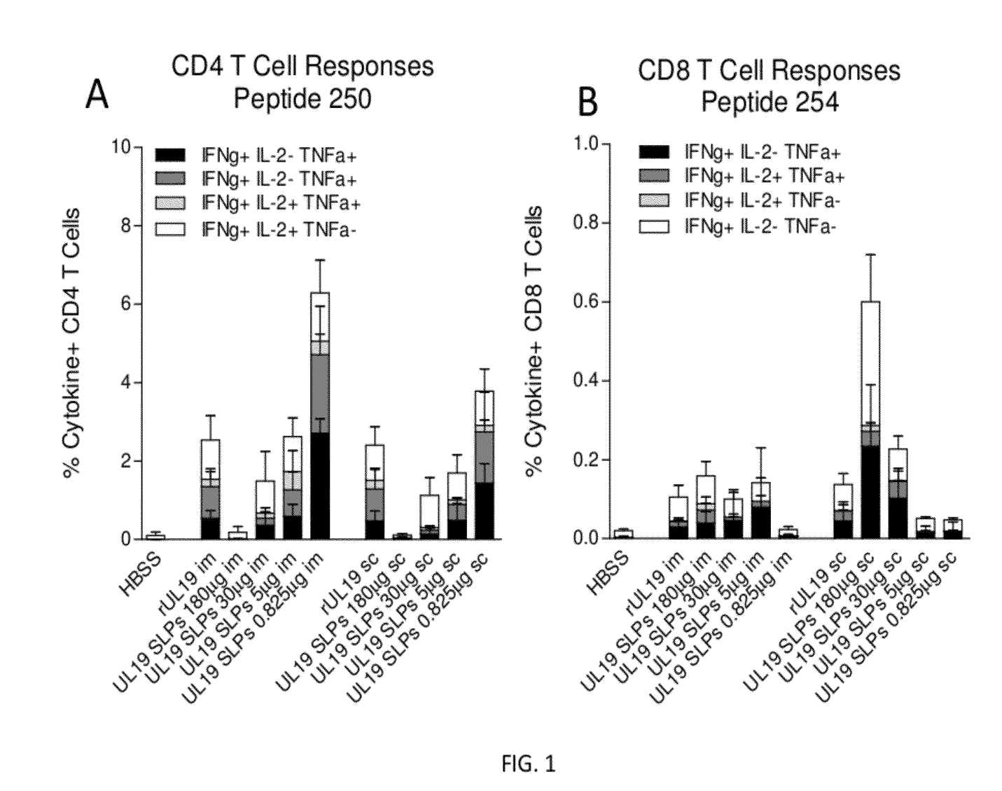

Surprisingly, as shown in FIG. 1, vaccination of mice with less than 1 μg of total SLP adjuvanted with GLA-SE induced CD4 responses specific for defined HSV-2 UL19 epitopes that were comparable or better than vaccination with 5 μg of UL19 full length recombinant protein. These data indicate that SLPs formulated in adjuvant can induce a robust cellular immune response in mice at a substantially low antigen dose.

The results thus demonstrate that the use of SLPs+GLA-SE in a prime/boost vaccine regimen is effective at generating antigen-specific Th1 CD4 T-cell responses (both i.m. & s.c. delivery). The observed Th1 CD4 T-cell responses were dependent on SLP dose: Th1 CD4 T-cell responses increased with decreasing SLP dose. Th1 CD4 T-cell responses were equal or greater than to those observed using recombinant protein+GLA-SE when SLPs were delivered at a molar dose equivalent. The results also demonstrate that the use of SLPs+GLA-SE in a prime/boost vaccine regimen is effective at generating antigen-specific CD8 T-cell responses (s.c. delivery is superior to i.m.). The observed CD8 T-cell responses were dependent on SLP dose: CD8 T-cell responses increased with increasing SLP dose.

### EXAMPLE 2

Vaccination with SLPs+GLA-SE is Effective at Generating Antigen-Specific CD4 and CD8 T-cell Responses Against SIV-Gag

This Example addresses the question of whether CD8/CD4 T-cell responses directed against SW-Gag can be observed when SIV-Gag is delivered as recombinant protein or at varying doses of SLP as measured by ex vivo restimulation of splenocytes with peptides previously identified to contain the SW-Gag CD8 (AL11) or CD4 (DD13) T-cell epitopes.

The amino acid sequence for SIV-Gag 289-333 is as follows:

GPKEPFQSYVDRFYKSLRAEQTDAAVKNWMTQTLLIQNANPDCKL (SEQ ID NO: 18). [The CD4 T-cell epitope DD13 (DRFYKSLRAEQTD, corresponding to amino acids 299-311 of SIV-Gag; SEQ ID NO: 19) is underlined while the CD8 T-cell epitope AL11(AAVKNWMTQTL, corresponding to amino acids 312-322 of SIV-Gag; SEQ ID NO: 20) is shown in bold].

Various doses of SLPs or recombinant full length SIV-Gag protein were formulated with GLA-SE (5 μg GLA in 2% SE (i.e., 2 wt % oil in an oil-in-water emulsion)) as shown in Table D. Samples were brought to the final volumes for injection in PBS. The amount of antigen in a 50 !IL dose is shown in Table D.

TABLE D

| | Total | Dose Relationship to |
|---|---|---|
| | Antigen | aa Sequence in |
| Antigen | Dose | Native rP (molar) |
| rSIV-Gag protein | 5 μg | NA |
| 1, SIV-Gag SLP | 45 μg | 100x |
| 1, SIV-Gag SLP | 4.5 μg | 10x |
| 1, SIV-Gag SLP | 0.45 μg | equimolar |
| 1, SIV-Gag SLP | 0.045 μg | 0.1x |
| rSIV-Gag protein | 10 μg | NA |
| 1, SIV-Gag SLP | 90 μg | 100x |
| 1, SIV-Gag SLP | 9.0 μg | 10x |
| 1, SIV-Gag SLP | 0.9 μg | equimolar |
| 1, SIV-Gag SLP | 0.09 μg | 0.1x |

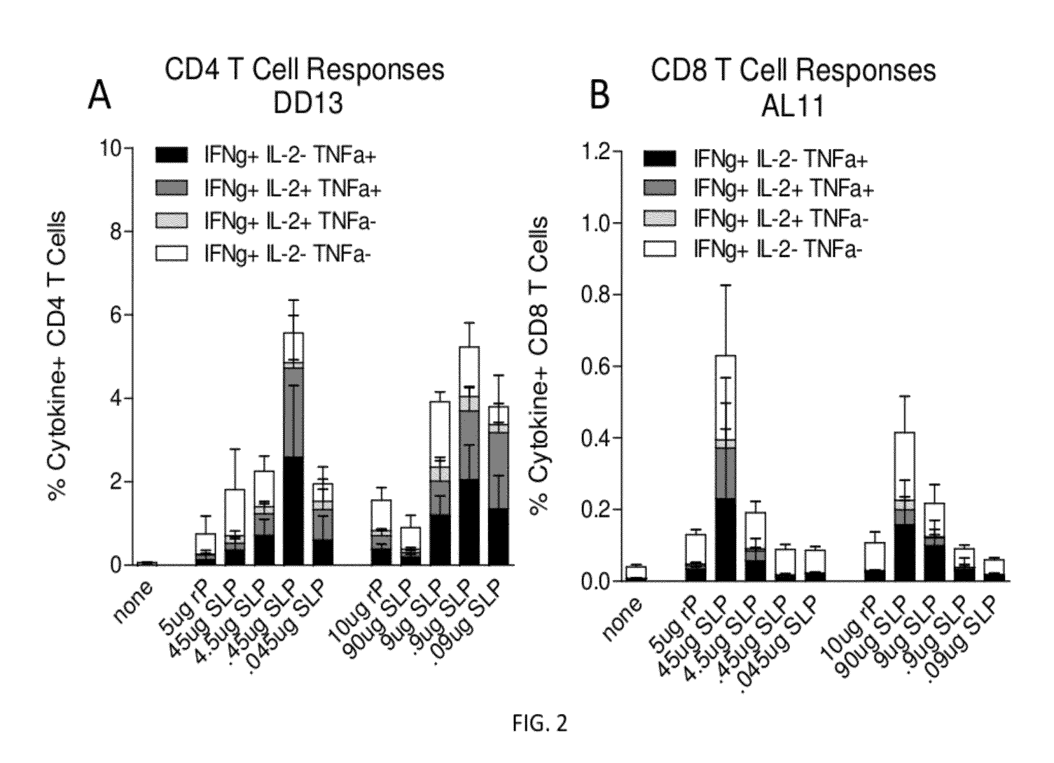

Female C57BL/6 mice mice were immunized with 50 μL of the antigen formulations identified in Table D by subcutaneous (s.c) injection at the base of the tail. On day 21 post-immunization, mice were administered a second dose of vaccine (boost) identical to the first dose (prime). Antigen-specific splenic CD4 and CD8 T-cell responses were measured on day 5 post-boost. The results are shown in FIG. 2.

The results in FIG. 2 demonstrate that the use of SLPs+GLA-SE in a prime/boost vaccine regimen is effective at generating antigen-specific Th1 CD4 T-cell responses (s.c. delivery). The observed Th1 CD4 T-cell responses were dependent on SLP dose: Th1 CD4 T cell responses increased with decreasing SLP dose (down to 0.09 ug). Th1 CD4 T-cell responses were greater than to those observed using recombinant protein+GLA-SE when SLPs were delivered at a molar dose equivalent. Th1 CD4 T-cell responses were greater than or equal to those observed using recombinant protein+GLA-SE when SLPs were delivered over a three log dose range that included a molar dose equivalent. The results also demonstrate that the use of SLPs+GLA-SE in a prime/boost vaccine regimen is effective at generating antigen-specific CD8 T cell responses (s.c. delivery). The observed CD8 T-cell responses were dependent on SLP dose: CD8 T-cell responses increased with increasing SLP dose.

### EXAMPLE 3

### Importance of Adjuvant for Generating Antigen-Specific CD4 and CD8 T-Cell Responses

This Example addresses the question of whether (1) CD8/CD4 T-cell responses directed against SW-Gag can be observed when SIV-Gag is delivered as recombinant protein or at varying doses of SLP as measured by ex vivo restimulation of splenocytes with peptides previously identified to contain the SW-Gag CD8 (AL11) or CD4 (DD13) T-cell epitopes, and (2) whether an adjuvant is necessary to observe these responses.

Various doses of SLPs or recombinant full length SIV-Gag protein were formulated with GLA-SE (5 μg GLA in 2% SE (i.e., 2 wt % oil in an oil-in-water emulsion)) or 2% SE (without GLA) as shown in Table E. Samples were brought to the final volumes for injection in PBS. The amount of antigen in a 50 μL dose is shown in Table E.

TABLE E

| | | Dose Relationship |
|---|---|---|
| | Total Antigen | to aa Sequence in |
| Antigen | Dose | Native rP (molar) |
| rSIV-Gag protein | 10 μg | NA |
| SIV-Gag SLP | 90 μg | 100x |
| SIV-Gag SLP | 9.0 μg | 10x |
| SIV-Gag SLP | 0.9 μg | equimolar |
| SIV-Gag SLP | 0.09 μg | 0.1x |
| SIV-Gag SLP | 0.009 μg | 0.01x |

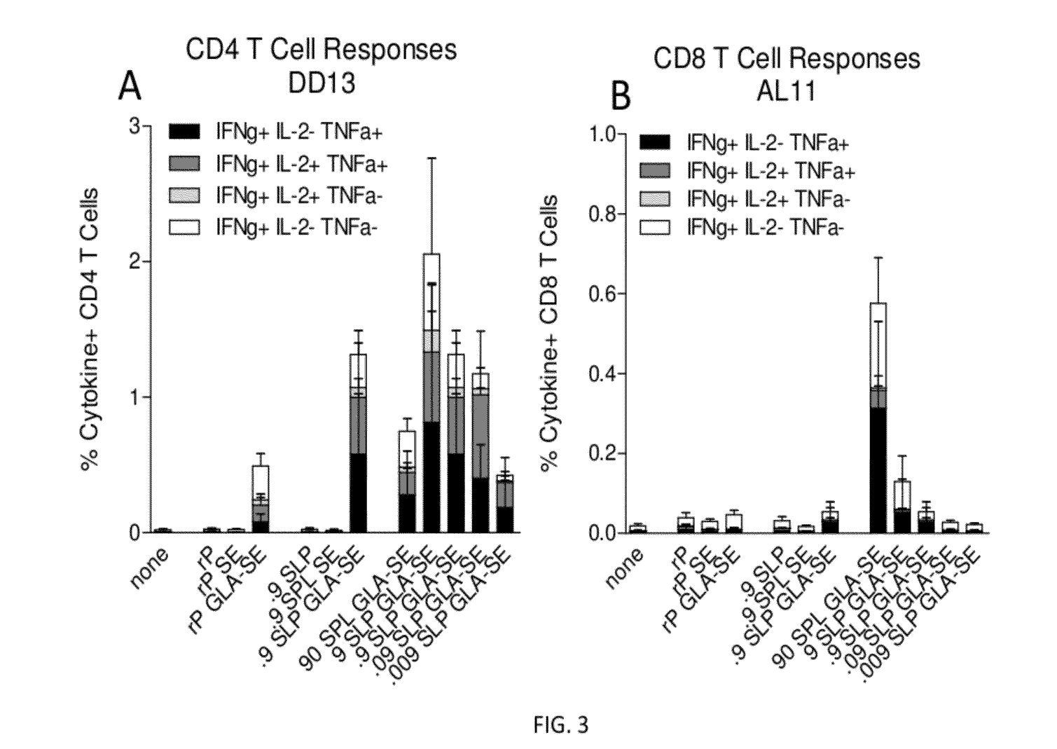

Female C57BL/6 mice mice were immunized with 50 μL of the antigen formulations identified in Table E by intramuscular (i.m.) injection in one hind limb. On day 21 post-immunization, mice were administered a second dose of vaccine (boost) identical to the first dose (prime). Antigen-specific splenic CD4 and CD8 T-cell responses were measured on day 6 post-boost. Mice were bled for serum to measure antibody responses. The results are shown in FIG. 3.

The results in FIG. 3 demonstrate that the use of SLPs+GLA-SE in a prime/boost vaccine regimen is effective at generating antigen-specific Th1 CD4 T-cell responses (i.m. delivery). The observed Th1 CD4 T-cell responses were dependent on SLP dose: Th1 CD4 T-cell responses were greater than or equal to those observed using recombinant protein+GLA-SE when SLPs were delivered over a four log dose range that included a molar dose equivalent. The results also show that Th1 CD4 T cell responses are dependent on the inclusion of an adjuvant. FIG. 3 also demonstrates that the use of SLPs+GLA-SE in a prime/boost vaccine regimen is effective at generating antigen-specific CD8 T-cell responses (i.m. delivery). Observed CD8 T-cell responses are dependent on SLP dose: CD8 T cell responses increased with increasing SLP dose. The results also show that CD8 T cell responses are dependent on the inclusion of an adjuvant.

### EXAMPLE 4

### Importance of Adjuvant During the Prime and/or Boost for Generating Antigen-Specific CD4 T-Cell Response

This Example addresses the question of whether adjuvant is required during the prime and the boost of an immunization regimen to generate antigen specific CD4 T-cell responses.

SLPs were formulated with GLA-SE (5 μg GLA in 2% SE (i.e., 2 wt % oil in an oil-in-water emulsion)), 2% SE (without GLA), or alone as shown in Table F. Samples were brought to the final volumes for injection in PBS.

TABLE F

| | | Boost |
|---|---|---|
| | Prime | (d21 post-prime) |
| SR01-040-1 | — | — |
| SR01-040-2 | SLP | SLP |
| SR01-040-3 | SLP | SLP + SE |
| SR01-040-4 | SLP | SLP + GLA-SE |
| SR01-040-5 | SLP + SE | SLP |
| SR01-040-6 | SLP + SE | SLP + SE |
| SR01-040-7 | SLP + SE | SLP + GLA-SE |
| SR01-040-8 | SLP + GLA-SE | SLP |
| SR01-040-9 | SLP + GLA-SE | SLP + SE |
| SR01-040-10 | SLP + GLA-SE | SLP + GLA-SE |

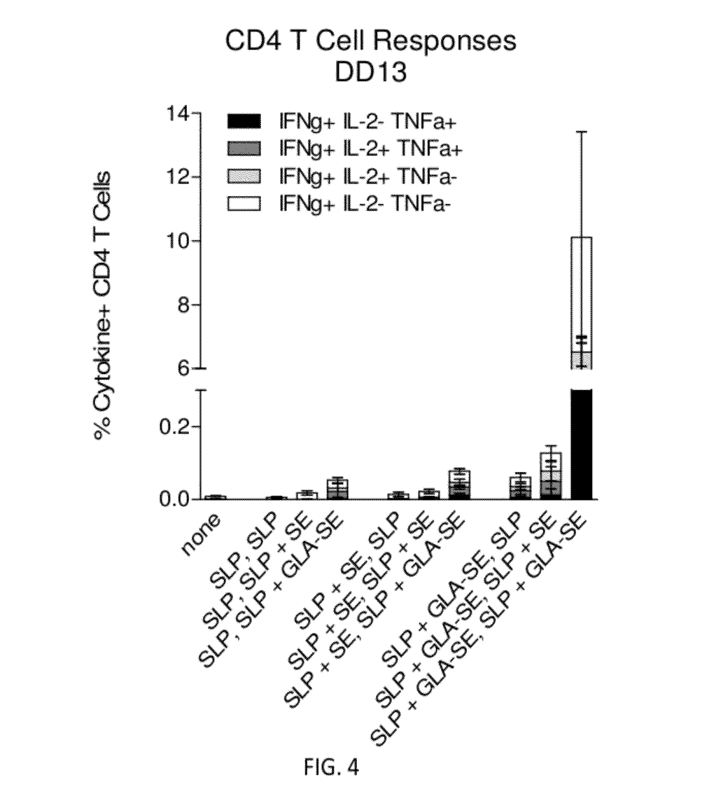

Female C57BL/6 mice were immunized with 50 μL of the antigen formulations identified in Table E by intramuscular (i.m.) injection in one hind limb. On day 21 post-immunization, mice were administered a second dose of vaccine (boost) according to Table F. Antigen-specific splenic CD4 T-cell responses were measured on day 5 post-boost. The results are shown in FIG. 4.

FIG. 4 demonstrates that the use of SLPs+GLA-SE in a prime/boost vaccine regimen is effective at generating antigen-specific Th1 CD4 T-cell responses (i.m. delivery). The results also show that the observed Th1 CD4 T-cell responses are dependent on the inclusion of an adjuvant during both the priming and boosting immunization.

From the foregoing it will be appreciated that, although specific embodiments have been described herein for purposes of illustration, various modifications may be made without deviating from the spirit and scope of the invention. Accordingly, the invention is not limited except as by the appended claims.

We claim:

1. A immunogenic composition comprising one or more SLPs (synthetic long peptides) and an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons.

2. The composition of claim 1, wherein the one or more SLPs are present at a concentration wherein an increase in the concentration results in a decrease in the immunogenicity of the composition.

3. The composition of claim 1, which comprises from about 0.1 μg to about 15 μg of each SLP.

4. The composition of claim 1, wherein the one or more SLPs are derived from an HSV-2 protein.

5. The composition of claim 4, wherein the HSV-2 protein is UL19.

6. The composition of claim 1, wherein the one or more SLPs are derived from a cancer antigen.

7. The composition of claim 6, wherein the cancer antigen is NY-ESO-1, TRP2, or CAIX.

8. The composition of claim 1, wherein the adjuvant is GLA.

9. The composition of claim 1, wherein the composition is aqueous and oil-free or comprises less than about 1% v/v oil.

10. The composition of claim 1, wherein the adjuvant is formulated in stable oil-in-water emulsion.

11. A method of immunizing a subject against HSV-2 comprising administering an immunogenic composition comprising one or more SLPs derived from an HSV-2 protein and an adjuvant, wherein the adjuvant comprises a disaccharide having a reducing and a non-reducing terminus each independently selected from glucosyl and amino substituted glucosyl, where a carbon at a 1 position of the non-reducing terminus is linked through either an ether (—O—) or amino (—NH—) group to a carbon at a 6′ position of the reducing terminus, the disaccharide being bonded to a phosphate group through a 4′ carbon of the non-reducing terminus and to a plurality of lipid groups through amide (—NH—C(O)—) and/or ester (—O—C(O)—) linkages, where the carbonyl (—C(O)—) group of the ester or amide linkage is directly linked to the lipid group, and each lipid group comprises at least 8 carbons.

12. The method of claim 11, wherein the adjuvant is GLA and the HSV-2 protein is UL19.

13. The method of claim 11, wherein a second composition comprising a immunogen is administered following administration of the SLP composition and wherein the immunogen is recombinantly produced.


SEQUENCE LISTING (20 sequences)

SEQ ID NO: 1 - protein, 1374 aa, Herpes Simplex Virus 2
MAAPARDPPGYRYAAAMVPTGSILSTIEVASHRRLFDFFARVRSDENSLYDVEFDALLGSYCNTLSLVRFLELGLSVACVCTKFPELAYMNEGRVQFEVHQPLIARDGPHPVEQPVHNYMTKVIDRRALNAAFSLATEAIALLTGEALDGTGISLHRQLRAIQQLARNVQAVLGAFERGTADQMLHVLLEKAPPLALLLPMQRYLDNGRLATRVARATLVAELKRSFCDTSFFLGKAGHRREAIEAWLVDLTTATQPSVAVPRLTHADTRGRPVDGVLVTTAAIKQRLLQSFLKVEDTEADVPVTYGEMVLNGANLVTALVMGKAVRSLDDVGRHLLEMQEEQLEANRETLDELESAPQTTRVRADLVAIGDRLVFLEALEKRIYAATNVPYPLVGAMDLTFVLPLGLFNPAMERFAAHAGDLVPAPGHPEPRAFPPRQLFFWGKDHQVLRLSMENAVGTVCHPSLMNIDAAVGGVNHDPVEAANPYGAYVAAPAGPGADMQQRFLNAWRQRLAHGRVRWVAECQMTAEQFMQPDNANLALELHPAFDFFAGVADVELPGGEVPPAGPGAIQATWRVVNGNLPLALCPVAFRDARGLELGVGRHAMAPATIAAVRGAFEDRSYPAVFYLLQAAIHGSEHVFCALARLVTQCITSYWNNTRCAAFVNDYSLVSYIVTYLGGDLPEECMAVYRDLVAHVEALAQLVDDFTLPGPELGGQAQAELNHLMRDPALLPPLVWDCDGLMRHAALDRHRDCRIDAGGHEPVYAAACNVATADFNRNDGRLLHNTQARAADAADDRPHRPADWTVHHKIYYYVLVPAFSRGRCCTAGVRFDRVYATLQNMVVPEIAPGEECPSDPVTDPAHPLHPANLVANTVNAMFHNGRVVVDGPAMLTLQVLAHNMAERTTALLCSAAPDAGANTASTANMRIFDGALHAGVLLMAPQHLDHTIQNGEYFYVLPVHALFAGADHVANAPNFPPALRDLARHVPLVPPALGANYFSSIRQPVVQHARESAAGENALTYALMAGYFKMSPVALYHQLKTGLHPGFGFTVVRQDRFVTENVLFSERASEAYFLGQLQVARHETGGGVSFTLTQPRGNVDLGVGYTAVAATATVRNPVTDMGNLPQNFYLGRGAPPLLDNAAAVYLRNAVVAGNRLGPAQPLPVFGCAQVPRRAGMDHGQDAVCEFIATPVATDINYFRRPCNPRGRAAGGVYAGDKEGDVIALMYDHGQSDPARPFAATANPWASQRFSYGDLLYNGAYHLNGASPVLSPCFKFFTAADITAKHRCLERLIVETGSAVSTATAASDVQFKRPPGCRELVEDPCGLFQEAYPITCASDPALLRSARDGEAHARETHFTQYLIYDASPLKGLSL

SEQ ID NO: 2 - protein, 506 aa, Simian immunodeficiency virus
MGARNSVLSGKKADELEKIRLRPGGKKKYMLKHVVWAANELDRFGLAESLLENKEGCQKILSVLAPLVPTGSENLKSLYNTVCVIWCIHAEEKVKHTEEAKQIVQRHLVVETGTAETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMAEALKEALAPVPIPFAAAQKRGPRKPIKCWNCGKEGHSARQCRAPRRQGCWKCGKMDHVMAKCPDRQAGFLGLGPWGKKPRNFPMAQVHQGLTPTAPPEDPAVDLLKNYMQLGKQQRESREKPYKEVTEDLLHLNSLFGGDQ

SEQ ID NO: 3 - protein, 15 aa, Herpes Simplex Virus 2
IQNGEYFYVLPVHAL

SEQ ID NO: 4 - protein, 15 aa, Herpes Simplex Virus 2
EYFYVLPVHALFAGA

SEQ ID NO: 5 - protein, 15 aa, Herpes Simplex Virus 2
NYFSSIRQPVVQHAR

SEQ ID NO: 6 - protein, 15 aa, Herpes Simplex Virus 2
SIRQPVVQHARESAA

SEQ ID NO: 7 - protein, 15 aa, Herpes Simplex Virus 2
CEFIATPVATDINYF

SEQ ID NO: 8 - protein, 15 aa, Herpes Simplex Virus 2
LGLSVACVCTKFPEL

SEQ ID NO: 9 - protein, 15 aa, Herpes Simplex Virus 2
VACVCTKFPELAYMN

SEQ ID NO: 10 - protein, 15 aa, Herpes Simplex Virus 2
SAAGENALTYALMAG

SEQ ID NO: 11 - protein, 15 aa, Herpes Simplex Virus 2
ENALTYALMAGYFKM

SEQ ID NO: 12 - protein, 15 aa, Herpes Simplex Virus 2
HPGFGFTVVRQDRFV

SEQ ID NO: 13 - protein, 45 aa, Herpes Simplex Virus 2
VPRRAGMDHGQDAVCEFIATPVATDINYFRRPCNPRGRAAGGVYA

SEQ ID NO: 14 - protein, 45 aa, Herpes Simplex Virus 2
GVLLMAPQHLDHTIQNGEYFYVLPVHALFAGADHVANAPNFPPAL

SEQ ID NO: 15 - protein, 45 aa, Herpes Simplex Virus 2
KMSPVALYHQLKTGLHPGFGFTVVRQDRFVTENVLFSERASEAYF

SEQ ID NO: 16 - protein, 45 aa, Herpes Simplex Virus 2
SYCNTLSLVRFLELGLSVACVCTKFPELAYMNEGRVQFEVHQPLI

SEQ ID NO: 17 - protein, 45 aa, Herpes Simplex Virus 2
PALGANYFSSIRQPVVQHARESAAGENALTYALMAGYFKMSPVAL

SEQ ID NO: 18 - protein, 45 aa, Simian immunodeficiency virus
GPKEPFQSYVDRFYKSLRAEQTDAAVKNWMTQTLLIQNANPDCKL

SEQ ID NO: 19 - protein, 13 aa, Simian immunodeficiency virus
DRFYKSLRAEQTD

SEQ ID NO: 20 - protein, 11 aa, Simian immunodeficiency virus
AAVKNWMTQTL